(19) United States
(12) Patent Application Publication
(10) Pub. No.: US 2020/0129603 A1
(43) Pub. Date: Apr. 30, 2020

(54) MEDICAL TREATMENT METHOD WITH ADMINISTRATION OF DENDRITIC CELLS

(71) Applicant: MEDIZINISCHE HOCHSCHULE HANNOVER, Hannover (DE)

(72) Inventor: Thomas Wirth, Sehnde (DE)

(21) Appl. No.: 16/737,672

(22) Filed: Jan. 8, 2020

Related U.S. Application Data
(62) Division of application No. 15/306,339, filed on Oct. 24, 2016.

(30) Foreign Application Priority Data
Apr. 29, 2014 (EP) 14166480.5
Sep. 25, 2014 (EP) 14186355.5

(51) Int. Cl. A61K 39/00; A61K 45/06; A61K 39/395; C07K 16/28; A61K 35/15

(52) U.S. Cl. CPC A61K 39/0011; A61K 2039/5158; A61K 39/3955; C07K 16/2878; C07K 16/2809; A61K 35/15; C07K 2317/75; A61K 2039/545; A61K 2039/572; A61K 2039/505; A61K 2039/5154; A61K 2039/55561; A61K 2039/55555; A61K 2039/6056; A61K 45/06

(57) ABSTRACT

A medical treatment method includes administering to a recipient a first composition comprising dendritic cells (DC) which are immunologically compatible with the recipient and which are associated with a target antigen. The method also includes administering to the recipient a second composition comprising at least a portion of the target antigen in soluble form and a co-stimulatory antibody effective for activating T-cells and/or the dendritic cells (DC), wherein the second composition is administered at least 1 day subsequent to administration of the first composition. The dendritic cells are preferably autologous dendritic cells.

### PRIORITY CLAIM AND REFERENCE TO RELATED APPLICATION

This is a divisional of and claims priority under 35 U.S.C. §§ 120 and 121 from prior application Ser. No. 15/306,339, which was filed on Oct. 24, 2016, which application was a 35 U.S.C. 371 US National Phase and claims priority under 35 U.S.C. § 119, 35 U.S.C. 365(b) and all applicable statutes and treaties from prior PCT Application PCT/EP2015/059399, which was filed Apr. 29, 2015, which application claimed priority from European Application EP 14166480.5, which was filed Apr. 29, 2014 and from European Application EP 14186355.5, which was filed Sep. 25, 2014.

### FIELD

The present invention relates to a medical treatment method that includes administering to a recipient a first composition comprising dendritic cells (DC) which are immunologically compatible with the recipient and which are associated with a target antigen; and administering to the recipient a second composition comprising at least a portion of the target antigen in soluble form and a co-stimulatory antibody effective for activating T-cells and/or the dendritic cells (DC), wherein the second composition is administered at least 1 day subsequent to administration of the first composition. The method achieves a pharmaceutical combination of compositions for use in the treatment or prevention of a disease having cells bearing a target antigen, e.g. as a vaccine and to a method for vaccination of a mammal, especially of a human for raising a cellular immune response directed against cells of the mammalian recipient, especially human recipient, which cells express a target antigen. The target antigen can e.g. be an autoantigen like a malignant antigen, i.e. a tumour-specific antigen, or an alloantigen specific for an infecting agent, e.g. an antigen specific for a virus or for an intracellular bacterium. Preferably, the pharmaceutical combination of compositions and the method are for medical use in the treatment of tumour and/or for medical use in the treatment of infections by a virus or by intracellular bacteria.

The pharmaceutical combination of compositions comprises or consists of a first composition and a second composition, wherein the second composition is for administration to the mammalian, especially human recipient, subsequent to the administration of the first composition, e.g. the second composition is provided for administration at least 1 day, preferably 2 to 10 days, e.g. 7 days following administration of the first composition. Accordingly, the method of medical treatment comprises the administration of the first composition and the subsequent administration of the second composition to a recipient, e.g. at least one day, preferably 2 to 10 days subsequent to administration of the first composition. The method of treatment is effective in inducing an antigen-specific T-cell response in the recipient, which response is directed against cells bearing the target antigen, which preferably is an auto-antigen like a tumour-antigen. The pharmaceutical combination of compositions and the method of treatment using the combination, respectively, have the advantage of raising an effective antigen-specific T-cell response against cells bearing a target antigen that can be an alloantigen or an autoantigen, especially against a target antigen which is a malignant autoantigen, e.g. raising an antigen-specific T-cell response against cells bearing a tumour-antigen. A further advantage is that the pharmaceutical combination of compositions can raise an antigen-specific T-cell response within a comparatively short time, e.g. within 10 to 14 days following administration of the first composition. The antigen-specific T-cell response is a CD8++T-cell response, preferably in combination with a CD4+ T-cell response.

### BACKGROUND

US 2003/0077263 A1 describes the in vitro production of antigen-presenting dendritic cells for use as a vaccine adjuvant against tumour. CD34+ hematopoietic progenitor cells and stem cells were stimulated with granulocyte-macrophage colony stimulating factor (GM-CSF) for in vitro expansion and differentiation into dendritic cells that were contacted with an antigen or transfected with a gene encoding the antigen, and subsequently activated with a CD40-binding protein. These antigen-pulsed dendritic cells were reintroduced into the original donor of the CD34+ cells.

US 2006/0204509 A1 describes immunization of mice with dendritic cells coated with a peptide representing an epitope of Listerium monocytogenes, followed by a booster immunization with complete Listerium monocytogenes bacteria.

Ahonen et al., J. Exp. Med. 775-784 (2004) describe the immunization of na\ddot{v}e mice using concomitant administration of the alloantigen ovalbumin or its epitope peptide SIINFEKL with a TLR agonist and anti-CD40 antibody. The combination of the TLR agonist and anti-CD40 antibody with the antigen was found to induce expansion of antigen-specific CD8+ T-cells. No booster immunization is described.

Poly(I:C) (polyinosinic:polycytidylic acid) is a mismatched double-stranded RNA, one strand being comprised of polyinosinic acid, the other strand of polycytidylic acid. Poly(I:C) is known to interact with TLR3 (Toll-like receptor 3) and is used as an immuno stimulant, e.g. using the sodium salt of Poly(I:C) for simulating viral infections.

Ricupito et al., Cancer Research 3545-3554 (2013) describe a vaccine comprising as a first component antigen-pulsed dendritic cells (DC), and for subsequent administration as a boost a second component of the antigen in complete Freunds adjuvant. The antigen was Tag-IV, the immunodominant CTL epitope from the SV40 Tag antigen. Ricupito et al. conclude that a single administration of the antigen-primed DC is effective against tumours, whereas the boost does not sustain survival of tumour-specific T_{CM} cells and is rather detrimental to long-lived immune surveillance.

Badovinac et al., Nature Medicine, 748-756 (2005) describe a first composition which can be DC primed with the antigen LLO of Listerium monocytogenes, vaccinia virus expressing the LLO91-99 epitope of Listerium monocytogenes or syngeneic spleen cells coated with the LLO91-99 epitope of Listerium monocytogenes, and a second composition for boosting, consisting of virulent Listerium monocytogenes.

Pham et al., PNAS 12198-12203 (2010) describe immunisation using antigen-coated synthetic PLGA microspheres to replace antigen-coated DC as a first component of a vaccine, using hen ovalbumin (Ova) as the antigen. Following administration of the first component consisting of antigen-coated PLGA microspheres, a second component of virulent Listerium monocytogenes expressing Ova (virLM-Ova), or of full-length Ova-protein plus poly(I:C) plus anti-CD40 mAb was administered for boosting. Pham et al. conclude that the effect of the boost is based on the cross-priming against particulate antigen, because immunisation with twice the amount of soluble Ova did not prime a boostable CD8 T-cell response.

US 2011/0274653 A1 describes a conjugate of an anti-CD40 antibody and an antigen for immunisation in a composition containing a TLR agonist and optionally an anti-CD40 antibody. No boosting composition for subsequent administration is mentioned.

WO 2012/135132 A1 describes a fusion peptide of an antibody specific for a DC specific cell surface receptor and a HCV antigen, which optionally is used in combination with a TLR agonist. No boosting composition for subsequent administration is mentioned.

Capece et al., J. of Biomedicine and Biotechnology 1-17 (2012) describe various costimulatory and co-inhibitory pathways affecting anti-tumour immune responses and anergy of T-cells against tumour antigens.

Yan Ge et al., Biomedicine and Pharmacotherapy 487-492 (2010) describe immunisation by administration of immature DC and the agonistic anti-CD40 antibody 5C11.

Lapteva et al., Cancer Res. 10548-10537 (2008) describes improving vaccines based on DC by use of a fusion peptide of the cytoplasmatic domain of anti-CD40 and a synthetic ligand binding domain with a membrane targeting sequence for inducing the CD40 signal cascade in DC.

### SUMMARY OF THE INVENTION

A preferred medical treatment method includes administering to a recipient a first composition comprising dendritic cells (DC) which are immunologically compatible with the recipient and which are associated with a target antigen. The method further includes administering to the recipient a second composition comprising at least a portion of the target antigen in soluble form and a co-stimulatory antibody effective for activating T-cells and/or the dendritic cells (DC), wherein the second composition is administered at least 1 day subsequent to administration of the first composition.

The dendritic cells are preferably autologous dendritic cells.3The dendritic cells (DC) are preferably associated with the target antigen by being contacted with the target antigen or by being contacted with a nucleic acid sequence encoding the antigen. The second composition preferably contains a TLR3 agonist, TLR7 agonist, TLR4 agonist, TLR9 agonist or combinations of at least two of these. The co-stimulatory antibody effective for activating dendritic cells (DC) is preferably selected from the group consisting of anti-CD137 antibody, an anti-CD40 antibody, an anti-OX40 antibody, anti-ICOS antibody, an anti-CD27 antibody, an anti-CD28 antibody, an anti-GITR antibody, an anti-human GITR/AITR antibody, an anti-HVEM antibody, an anti-TIM1 antibody, an anti-TIM3 antibody, and mixtures of at least two of these. The TLR3 agonist is preferably Poly(I:C) and/or PolyICLC or a homologue thereof. The first composition is preferably free from an adjuvant.

### BRIEF DESCRIPTION OF THE DRAWINGS

The invention is described in greater detail by way of mouse experiments with reference to the figures, which show for different first and second compositions administered to experimental animals in

FIG. 1 flow cytometry results of peripheral blood with staining for CD11a at day −1 prior to administration of the second composition at a), c), e), g), and i) with restimulation with the target antigen (Ndufs1) and at b), d), f), h) and j) without restimulation (Control) with antigen,

FIG. 2 flow cytometry results of peripheral blood with staining for IFN gamma at day −1 prior to administration of the second composition at a), c), e), g), and i) with restimulation with the target antigen (Ndufs1) and at b), d), f), h) and j) without restimulation (Control) with antigen,

FIG. 3 a graphical representation of the proportion of malignant antigen-specific CD8+ T-cells from the results of FIG. 2,

FIG. 4 a)-j) flow cytometry results of peripheral blood with staining for highly CD11a-positive T-cells at day 7 following administration of the second composition,

FIG. 5 a graphical representation of the proportion of activated, i.e. highly CD11a-positive T-cells from the results of FIG. 4,

FIG. 6 flow cytometry results of peripheral blood with staining for IFN gamma-positive T-cells at day 7 following administration of the second composition at a), c), e), g), and i) with restimulation with the target antigen (Ndufs1) and at b), d), f), h) and j) without restimulation (Control) with antigen,

FIG. 7 a graphical representation of the proportion of IFN gamma-positive T-cells in activated T-cells from the results of FIG. 6,

FIG. 8 a graphical representation of the proportion of antigen-specific T-cells activated by different priming regimens,

FIG. 9 a) to f) FACS results for one exemplary experimental animal each at different priming regimens,

FIG. 10 the proportion of CD11a^{hi} CD8+ T-cells for different second compositions,

FIG. 11 the proportion of IFNγ-positive CD8+ T-cells for different second compositions,

FIG. 12 the in vivo reduction of tumour volume,

FIG. 13 a graphical representation of the proportion of T-cells in white blood cells (WBC) when stimulated by different compositions,

FIG. 14 a graphical representation of the proportion of antigen-specific T-cells in white blood cells when stimulated by the compositions used for FIG. 13,

FIG. 15 a graphical representation of the proportion of antigen-specific T-cells in the T-cell response when stimulated by the compositions used for FIG. 13,

FIG. 16 a graphical representation of IL-6 contained in CD8+ T-cells raised by the compositions of the invention and by virulent Listerium monocytogenes and by virulent LCM virus,

FIG. 17 a graphical representation of IFNγ contained in CD8+ T-cells raised by the compositions of the invention and by virulent Listerium monocytogenes and by virulent LCM virus,

FIG. 18 a graphical representation of TNFα contained in CD8+ T-cells raised by the compositions of the invention and by virulent Listerium monocytogenes and by virulent LCM virus, and in

FIG. 19 the effect of various TLR agonists in the second composition.

### DETAILED DESCRIPTION OF THE PREFERRED EMBODIMENTS

The invention provides methods for administration of pharmaceutical compositions suitable for effectively raising an immune response, preferably a cellular immune response, especially a CD8+ T-cell response, against cells of a human recipient, which cells bear a target antigen, which preferably is a malignant antigen, e.g. a tumour antigen, preferably raising a CD8+ T-cell response against cancer.

Methods provide administration of a pharmaceutical combination of compounds, which are provided in a first composition of compounds and in a second composition of compounds, for medical use in the treatment or prevention especially of tumours or of infections by virus or intracellular bacteria. The pharmaceutical combination of compounds is provided for administration to a mammal, preferably a human, herein also referred to as a recipient, preferably a human recipient. The first composition is prepared for first administration and the second composition is prepared for separate administration subsequent to administration of the first composition, e.g. for administration subsequent to administration of the first composition by at least 1 to at least 10 days, e.g. subsequent by 2 to 7 days. The pharmaceutical combination of compounds, which are comprised in the first and second compositions, is adapted for eliciting a target antigen-specific CD8+ T-cell response, preferably including a target antigen-specific CD4+ T-cell response, for the prevention and/or treatment of cells bearing the target antigen, which preferably is a malignant antigen, e.g. an autologous tumour antigen for the prevention and/or treatment of tumours bearing tumour antigen. Alternatively, the target antigen is an alloantigen, e.g. an antigen caused by infection by intracellular pathogens, e.g. infections by intracellular bacteria or viral infections and the pharmaceutical combination of compounds is for use in the prevention and/or treatment of infections by a virus or by intracellular bacteria. Accordingly, the pharmaceutical combination is customized for vaccination or for the prevention and/or treatment of tumours or for the prevention and/or treatment of these infections. Generally, the first composition can also be termed a first component and the second composition can also be termed a second component, and the combination comprising or consisting of the first and second component can be termed a medicament or vaccine. Accordingly, the pharmaceutical combination of compositions for use in medical treatment comprises the combination of a first composition comprising professional antigen presenting cells (APC) which are dendritic cells (DC) which preferably are immunologically compatible with the recipient and which are associated with a target antigen, and a second composition comprising at least a portion of the target antigen in soluble form and a co-stimulatory antibody effective for activating T-cells and/or the dendritic cells (DC), wherein the second composition is for administration at a time at least 1 day, preferably 3 to 7 or up to 5 days subsequent to administration of the first composition.

Generally, the treatment and the combination of compositions, respectively, can be for neoadjuvant use, e.g. for reduction of the tumour prior to surgery, and/or for adjuvant use, e.g. following tumour surgery, or for palliative use, e.g. without tumour surgery.

The pharmaceutical combination of compounds can be provided for use in the treatment of tumours that can be selected from the group comprising solid cancers and hematological malignancies, e.g. selected from the group comprising or consisting of hematological malignancies, e.g. Hodgkin and non-Hodgkin lymphomas, leukemia, e.g. acute lymphoblastic leukemia, acute myeloid leukemia, chronic lymphocytic leukemia, chronic myeloid leukemia, monocytic leukemia, myelomas, myeloproliferative diseases, myelodysplastic syndromes and solid cancers, e.g. originating from brain, head and neck, lung, pleura, heart, liver, kidney, colon, pancreas, stomach, gut, urinary tract, prostate, uterus, ovaries, breast, skin, testes, larynx and sarcoma.

Further, the invention relates to a method of treatment, raising a target antigen specific immune response, especially a cellular immune response specifically directed against cells bearing a target antigen, which especially is a malignant or a tumour antigen, preferably a homologous antigen, e.g. human tumour antigen, or a viral antigen or an intracellular bacterial antigen, by administration of the components comprised in the first composition and in the second composition of the pharmaceutical combination to a recipient. Further, the invention relates to the use of the pharmaceutical combination of the first and second compositions in the production of a medicament for the prevention and/or treatment of infection by intracellular pathogens, e.g. infections by intracellular bacteria or viral infections, or for the prevention and/or treatment of tumours, especially of tumours bearing tumour-specific antigen. Accordingly, the invention also relates to a method of prevention and/or treatment of infection by intracellular pathogens, e.g. infections by intracellular bacteria or viral infections, or for the prevention and/or treatment of tumours, especially of tumours bearing tumour-specific antigen.

The first composition of the pharmaceutical combination is customised for priming a target antigen-specific CD8+ T-cell response, preferably inducing the generation of target antigen-specific memory T-cells, and the second composition is customised for boosting the target antigen-specific CD8+ T-cell response. It has been found that the second composition can be customised for administration 2 to 10 days, e.g. 5 to 7 days following administration of the first composition for generating an important antigen-specific CD8+ T-cell response or cell number. Currently it is assumed that target antigen-specific memory T-cells, especially those specific for a target antigen that is a tumour antigen, are induced within at maximum 10, preferably at maximum 7 days following administration of the first composition, and accordingly, the memory T-cells induced by the administration of the combination of compounds of the invention can be described as early memory T-cells. Generally, it is preferred that the administration of the first composition does not induce a systemic inflammation. Accordingly, the target antigen contained in the first composition preferably is an autoantigen, e.g. a tumour antigen, and/or the first composition is free from adjuvants that stimulate an immune response.

Preferably, the immune response additionally induces target antigen-specific CD4+ T-cells that support B-cell mediated antibody production and tumour-specific Th1 T-cell responses. Further, the invention relates to a method for raising an antigen-specific T-cell response in a recipient against cells expressing a target antigen by administration of the pharmaceutical combination, firstly administration of the first composition, and subsequently of the second composition, with a temporal delay of at least 1 day.

The first composition comprises professional antigen presenting cells (APC), which APC preferably express MHC I, preferably dendritic cells (DC), the APC preferably in addition expressing MHC II. Dendritic cells (DCs) are identified by at least one, preferably all of the following surface markers: CD1a, CD1b, CD1c, CD4, CD11c, CD33, CD40, CD80, CD86, CD83, and HLA-DR. DCs include dendritic cell precursor cells, having at least one, preferably all of the following cell surface markers: CD123, CD45RA, CD36, and CD4. The APC are immunologically compatible with the recipient of the pharmaceutical combination, preferably autologous APC, which APC are loaded with the target antigen. The target antigen is an antigen specific for the malignant cells within the recipient, e.g. selected from tumour-specific antigens (malignant antigens), viral antigens and antigens of intracellular bacteria. The APC can be loaded with the target antigen by in vitro contact with the target antigen and/or, for proteinaceous antigen, by in vitro introduction of a nucleic acid sequence encoding the target antigen, e.g. by in vitro transduction or transfection of a nucleic acid sequence encoding the target antigen in an expression cassette.

The APC can originate from the recipient or from an immunologically compatible mammal, preferably a human, e.g. by isolation from peripheral blood. Optionally, the APC can be propagated in vitro by cultivation prior to or following loading with the malignant antigen. For example, APC, e.g. DC can be monocyte-derived DCs or isolated DCs after in vivo induction of DCs. Monocyte-derived DCs can be generated from autologous blood by isolation of monocytes, optional cultivation of monocytes, and differentiation to dendritic cells. In vivo induction of DCs can be obtained by administration of DC growth stimuli, e.g. of flt3 ligand, followed by isolation of DCs, e.g. from peripheral blood.

The first composition can contain the APC which are loaded with a target antigen in a pharmaceutically acceptable formulation that is adapted to keep intact the antigen-loaded APC. Preferably, the first composition is a formulation for intramuscular, sub-cutaneous, intra-venous or intraperitoneal administration. An exemplary formulation of the first composition comprises or consists of immunologically compatible APC loaded with a malignant antigen suspended in physiologic solution, e.g. physiologic saline.

The effective induction of a target antigen-specific immune response by the combination of the first and the second composition, which target antigen preferably is a tumour antigen, which is autologous, is surprising, because tumours generally evade the immune surveillance and express homologous antigen, against which generally immune responses of only very low magnitude can be elicited. Further, it is surprising that a high number of target antigen-specific T-cells can be induced by administration of the second composition within a short time subsequent to administration of the first composition. The short time delay between administration of the first and of the second compositions allow the use of the combination of the compositions for use in the treatment of tumours or in the treatment of infections by virus or intracellular bacteria, because no long time delay needs to occur before an effective target antigen-specific cellular immune response is induced.

In the alternative to APC loaded with the target antigen, the first composition can comprise the target antigen coupled to an antibody specific for the APC, especially coupled to an antibody specific for a DC surface receptor, e.g. anti-DEC205 antibody or anti-DCIR antibody. In this embodiment, the conjugate comprising the malignant antigen coupled to the antibody specific for the APC following administration of the first composition to the recipient results in the conjugate binding to APC within the recipient and generating APC loaded with the target antigen.

In the embodiments of the invention, the second composition comprises or consists of a target antigen and a co-stimulatory antibody for CD8+-T cells and/or for DC, and preferably a TLR3 agonist, e.g. Poly(I:C) or PolyICLC (polyinosinic-polycytidylic acid stabilized with polylysine and carboxymethylcellulose, available under the trademark Hiltonol), a TLR7 agonist, a TLR4 agonist, a TLR9 agonist and combinations of at least two of these, in a pharmaceutical formulation. The co-stimulatory agonistic antibody for CD8+ T-cells and/or DC is a molecule specifically directed against a surface receptor of T-cells and/or of DCs of the recipient and can e.g. be selected from an anti-CD137 antibody, an anti-CD40 antibody, an anti-OX40 antibody, an anti-ICOS antibody, an anti-CD27 antibody, an anti-CD28 antibody, an anti-GITR antibody, specifically anti-human GITR/AITR antibody, an anti-HVEM antibody, an anti-TIM1 antibody, an anti-TIM3 antibody, and mixtures of these. The second composition is a formulation for intramuscular, sub-cutaneous, intra-venous or intraperitoneal administration. Optionally, the second composition is provided for systemic administration. The second composition can be provided for administration, e.g. injection, at the same site or at a different site of the recipient's body.

The target antigens of the first composition and of the second composition contain at least one identical epitope for MHC I. Preferably, the malignant antigen of the first composition and the malignant antigen of the second composition share at least one section, the section having an identity of at least 80%, preferably for at least 90%, more preferably for at least 95% or at least 99% of the amino acid sequence. Optionally, the malignant antigens of the first composition and of the second composition are identical.

The malignant antigen preferably is soluble in aqueous media, e.g. in medium containing DCs and/or first composition and in the second composition, and preferably is a proteinaceous antigen, e.g. comprising at least 8 amino acids. Generally, the tumour antigen can be an antigen that is re-expressed in tumour cells or an antigen that is overexpressed in tumour cells, e.g. in comparison to differentiated normal cells. Exemplary tumour antigens of humans are telomerase, oncofetal proteins, e.g. alpha feto protein, and testis antigen, e.g. NY-ESO-1. For example, a tumour specific antigen is one of the group comprising or consisting of tumour antigens resulting from mutations, shared tumour antigens, differentiation antigens, antigens overexpressed in tumours, especially Nras, Hras, Kras which are indicative of a neoplastic state, tumour-specific antigens of the MAGE (including MAGE-B5, MAGE-B6, MAGE, MAGE-C2, MAGE-C3, MAGE-D), HAGE, SAGE, SSX-2, BAGE, TRAG-3, and GAGE families, including NY-ESO-1, LAGE, CAMEL, as well as MUC1, most preferably tumour-specific mutant Ras, e.g. Nras, Nras G12V, or Kras, KrasG12D and other common mutations.

Exemplary tumour antigens resulting from mutations are for lung carcinoma FIASNGVKLV (SEQ ID NO: 1), for melanoma YSVYFNLPADTIYTN(SEQ ID NO: 2), for chronic myeloid leukemia SSKALQRPV (SEQ ID NO: 3) or GFKQSSKAL (SEQ ID NO: 4) or ATGFKQSSKALQRPVAS (SEQ ID NO: 5) , for melanoma EDLTVKIGDFGLATEKSRWSGSHQFEQLS (SEQ ID NO: 6), for colorectal, gastric, and endometrial carcinoma FLIIWQNTM (SEQ ID NO: 7), for head and neck squamous cell carcinoma FPSDSWCYF (SEQ ID NO: 8), for melanoma SYLDSGIHF (SEQ ID NO: 9), for melanoma FSWAMDLDPKGA (SEQ ID NO: 10), for melanoma ACDPHSGHFV(SEQ ID NO: 11), for melanoma AVCPWTWLR (SEQ ID NO: 12), for colorectal carcinoma TLYQDDTLTLQAAG (SEQ ID NO: 13) or TLYQDDTLTLQAAG (SEQ ID NO: 14), for myeloid leukemia TMKQICKKEIRRLHQY (SEQ ID NO: 15), for melanoma KILDAVVAQK (SEQ ID NO: 16), for lung squamous CC especially ETVSEQSNV (SEQ ID NO: 17), for acute lymphoblastic leukemia RIAECILGM (SEQ ID NO: 18) or IGRIAECILGMNPSR (SEQ ID NO: 19) or IGRIAECILGMNPSR (SEQ ID NO: 20), for acute myelogenous leukemia YVDFREYEYY (SEQ ID NO: 21), for melanoma MIFEKHGFRRTTPP (SEQ ID NO: 22), for melanoma TLDWLLQTPK (SEQ ID NO: 23), for melanoma WRRAPAPGA (SEQ ID NO: 24) or PVTWRRAPA (SEQ ID NO: 25), for renal cell carcinoma, for melanoma and renal cell carcinoma SLFEGIDIYT (SEQ ID NO: 26), for bladder tumour AEPINIQTW (SEQ ID NO: 27), for melanoma FLEGNEVGKTY (SEQ ID NO: 28), for non-small cell lung carcinoma FLDEFMEGV (SEQ ID NO: 29), for melanoma EEKLIVVLF (SEQ ID NO: 30), for melanoma SELFRSGLDSY (SEQ ID NO: 31) or FRSGLDSYV (SEQ ID NO: 32), for melanoma EAFIQPITR (SEQ ID NO: 33), for melanoma RVIKNSIRLTL (SEQ ID NO: 34), for melanoma KINKNPKYK (SEQ ID NO: 35), lung squamous cell carcinoma QQITKTEV (SEQ ID NO: 36), colorectal carcinoma SLYKFSPFPL (SEQ ID NO: 37), for melanoma KELEGILLL (SEQ ID NO: 38), for head and neck squamous cell carcinoma VVPCEPPEV (SEQ ID NO: 39), for promyelocytic leukemia NSNHVASGAGEAAIETQSSSSEEIV (SEQ ID NO: 40), for melanoma LLLDDLLVSI (SEQ ID NO: 41), for melanoma PYYFAAELPPRNLPEP (SEQ ID NO: 42), for pancreatic adenocarcinoma VVVGAVGVG (SEQ ID NO: 43), for melanoma ILDTAGREEY (SEQ ID NO: 44), for melanoma RPHVPESAF (SEQ ID NO: 45), for melanoma KIFSEVTLK (SEQ ID NO: 46), for melanoma SHETVIIEL (SEQ ID NO: 47), for sarcoma QRPYGYDQIM (SEQ ID NO: 48), for colorectal carcinoma RLSSCVPVA (SEQ ID NO: 49), for melanoma GELIGILNAAKVPAD (SEQ ID NO: 50).

Exemplary shared tumour antigens are:

(SEQ ID NO: 51)
AARAVFLAL,
(SEQ ID NO: 52)
YRPRPRRY,
(SEQ ID NO: 53)
YYWPRPRRY,
(SEQ ID NO: 54)
VLPDVFIRC(V),
(SEQ ID NO: 55)
MLAVISCAV,
(SEQ ID NO: 56)
RQKRILVNL,
(SEQ ID NO: 57)
NYNNFYRFL,
(SEQ ID NO: 58)
EYSKECLKEF,
(SEQ ID NO: 59)
EYLSLSDKI,
(SEQ ID NO: 60)
MLMAQEALAFL,
(SEQ ID NO: 61)
SLLMWITQC,
(SEQ ID NO: 62)
LAAQERRVPR,
(SEQ ID NO: 63)
ELVRRILSR,
(SEQ ID NO: 64)
APRGVRMAV,
(SEQ ID NO: 65)
SLLMWITQCFLPVF,
(SEQ ID NO: 66)
QGAMLAAQERRVPRAAEVPR,
(SEQ ID NO: 67)
AADHRQLQLSISSCLQQL,
(SEQ ID NO: 68)
CLSRRPWKRSWSAGSCPGMPHL,
(SEQ ID NO: 69)
CLSRRPWKRSWSAGSCPGMPHL,
(SEQ ID NO: 70)
ILSRDAAPLPRPG,
(SEQ ID NO: 71)
AGATGGRGPRGAGA,
(SEQ ID NO: 72)
EADPTGHSY,
(SEQ ID NO: 73)
KVLEYVIKV,
(SEQ ID NO: 74)
SLFRAVITK,
(SEQ ID NO: 75)
EVYDGREHSA,
(SEQ ID NO: 76)
RVRFFFPSL,
(SEQ ID NO: 77)
EADPTGHSY,
(SEQ ID NO: 78)
REPVTKAEML,
(SEQ ID NO: 79)
DPARYEFLW,
(SEQ ID NO: 80)
ITKKVADLVGF,
(SEQ ID NO: 81)
SAFPTTINF,
(SEQ ID NO: 82)
SAYGEPRKL,
(SEQ ID NO: 83)
SAYGEPRKL,
(SEQ ID NO: 84)
TSCILESLFRAVITK,
(SEQ ID NO: 85)
PRALAETSYVKVLEY,
(SEQ ID NO: 86)
FLLLKYRAREPVTKAE,
(SEQ ID NO: 87)
EYVIKVSARVRF,
(SEQ ID NO: 88)
YLQLVFGIEV,
(SEQ ID NO: 89)
EYLQLVFGI,
(SEQ ID NO: 90)
REPVTKAEML,
(SEQ ID NO: 91)
EGDCAPEEK,
(SEQ ID NO: 92)
LLKYRAREPVTKAE,
(SEQ ID NO: 93)
EVDPIGHLY,
(SEQ ID NO: 94)
FLWGPRALV,
(SEQ ID NO: 95)
KVAELVHFL,
(SEQ ID NO: 96)
TFPDLESEF,
(SEQ ID NO: 97)
VAELVHFLL,
(SEQ ID NO: 98)
MEVDPIGHLY,
(SEQ ID NO: 99)
EVDPIGHLY,
(SEQ ID NO: 100)
REPVTKAEML,
SEQ ID NO: 101)
AELVHFLLL,
(SEQ ID NO: 102)
MEVDPIGHLY,
(SEQ ID NO: 103)
WQYFFPVIF,
(SEQ ID NO: 104)
EGDCAPEEK,
(SEQ ID NO: 105)
KKLLTQHFVQENYLEY,
(SEQ ID NO: 106)
KKLLTQHFVQENYLEY,
(SEQ ID NO: 107)
ACYEFLWGPRALVETS,
(SEQ ID NO: 108)
VIFSKASSSLQL,
(SEQ ID NO: 109)
VIFSKASSSLQL,
(SEQ ID NO: 110)
GDNQIMPKAGLLIIV,
(SEQ ID NO: 111)
TSYVKVLHHMVKISG,
(SEQ ID NO: 112)
RKVAELVHFLLLKYRA,
(SEQ ID NO: 113)
FLLLKYRAREPVTKAE,
(SEQ ID NO: 114)
EVDPASNTY,
(SEQ ID NO: 115)
GVYDGREHTV,
(SEQ ID NO: 116)
NYKRCFPVI,
(SEQ ID NO: 117)
SESLKMIF,
(SEQ ID NO: 118)
MVKISGGPR,
(SEQ ID NO: 119)
EVDPIGHVY,
(SEQ ID NO: 120)
REPVTKAEML,
(SEQ ID NO: 121)
EGDCAPEEK,
(SEQ ID NO: 122)
ISGGPRISY,
(SEQ ID NO: 123)
LLKYRAREPVTKAE,
(SEQ ID NO: 124)
ALSVMGVYV,
(SEQ ID NO: 125)
GLYDGMEHL,
(SEQ ID NO: 126)
DPARYEFLW,
(SEQ ID NO: 127)
FLWGPRALV,
(SEQ ID NO: 128)
VRIGHLYIL,
(SEQ ID NO: 129)
EGDCAPEEK,
(SEQ ID NO: 130)
REPFTKAEMLGSVIR,
(SEQ ID NO: 131)
AELVHFLLLKYRAR,
(SEQ ID NO: 132)
LLFGLALIEV,
(SEQ ID NO: 133)
ALKDVEERV,
(SEQ ID NO: 134)
SESIKKKVL,
(SEQ ID NO: 135)
PDTRPAPGSTAPPAHGVTSA,
(SEQ ID NO: 136)
QGQHFLQKV,
(SEQ ID NO: 137)
SLLMWITQC,
(SEQ ID NO: 138)
MLMAQEALAFL,
(SEQ ID NO: 139)
ASGPGGGAPR,
(SEQ ID NO: 140)
LAAQERRVPR,
(SEQ ID NO: 141)
TVSGNILTIR,
(SEQ ID NO: 142)
APRGPHGGAASGL,
(SEQ ID NO: 143)
MPFATPMEA,
(SEQ ID NO: 144)
KEFTVSGNILTI,
(SEQ ID NO: 145)
MPFATPMEA,
(SEQ ID NO: 146)
LAMPFATPM,
(SEQ ID NO: 147)
ARGPESRLL,
(SEQ ID NO: 148)
SLLMWITQCFLPVF,
(SEQ ID NO: 149)
LLEFYLAMPFATPMEAELARRSLAQ,
(SEQ ID NO: 150)
LLEFYLAMPFATPMEAELARRSLAQ,
SEQ ID NO: 151)
EFYLAMPFATPM,
(SEQ ID NO: 152)
RLLEFYLAMPFA,
(SEQ ID NO: 153)
QGAMLAAQERRVPRAAEVPR,
(SEQ ID NO: 154)
PGVLLKEFTVSGNILTIRLT,
(SEQ ID NO: 155)
VLLKEFTVSG,
(SEQ ID NO: 156)
AADHRQLQLSISSCLQQL,
(SEQ ID NO: 157)
LLEFYLAMPFATPMEAELARRSLAQ,
(SEQ ID NO: 158)
LKEFTVSGNILTIRL,
(SEQ ID NO: 159)
PGVLLKEFTVSGNILTIRLTAADHR,
(SEQ ID NO: 160)
LLEFYLAMPFATPMEAELARRSLAQ,
(SEQ ID NO: 161)
AGATGGRGPRGAGA,
(SEQ ID NO: 162)
LYATVIHDI,
(SEQ ID NO: 163)
ILDSSEEDK,
(SEQ ID NO: 164)
KASEKIFYV,
(SEQ ID NO: 165)
EKIQKAFDDIAKYFSK,
(SEQ ID NO: 166)
WEKMKASEKIFYVYMKRK,
(SEQ ID NO: 167)
KIFYVYMKRKYEAMT,
(SEQ ID NO: 168)
KIFYVYMKRKYEAM,
(SEQ ID NO: 169)
INKTSGPKRGKHAWTHRLRE,
(SEQ ID NO: 170)
YFSKKEWEKMKSSEKIVYVY,
(SEQ ID NO: 171)
MKLNYEVMTKLGFKVTLPPF,
(SEQ ID NO: 172)
KHAWTHRLRERKQLVVYEEI,
(SEQ ID NO: 173)
LGFKVTLPPFMRSKRAADFH,
(SEQ ID NO: 174)
KSSEKIVYVYMKLNYEVMTK,
(SEQ ID NO: 175)
KHAWTHRLRERKQLVVYEEI,
(SEQ ID NO: 176)
SLGWLFLLL,
(SEQ ID NO: 177)
LSRLSNRLL,
(SEQ ID NO: 178)
LSRLSNRLL,
(SEQ ID NO: 179)
CEFHACWPAFTVLGE,
(SEQ ID NO: 180)
CEFHACWPAFTVLGE,
(SEQ ID NO: 181)
CEFHACWPAFTVLGE,
(SEQ ID NO: 182)
EVISCKLIKR
or
(SEQ ID NO: 183)
CATWKVICKSCISQTPG.

Exemplary tumour differentiation antigens are:

(SEQ ID NO: 184)
YLSGANLNL,
(SEQ ID NO: 185)
IMIGVLVGV,
(SEQ ID NO: 186)
GVLVGVALI,
(SEQ ID NO: 187)
HLFGYSWYK,
(SEQ ID NO: 188)
QYSWFVNGTF,
(SEQ ID NO: 189)
TYACFVSNL,
(SEQ ID NO: 190)
AYVCGIQNSVSANRS,
(SEQ ID NO: 191)
DTGFYTLHVIKSDLVNEEATGQFRV,
(SEQ ID NO: 192)
YSWRINGIPQQHTQV,
(SEQ ID NO: 193)
TYYRPGVNLSLSC,
(SEQ ID NO: 194)
EIIYPNASLLIQN,
(SEQ ID NO: 195)
YACFVSNLATGRNNS,
(SEQ ID NO: 196)
LWWVNNQSLPVSP,
(SEQ ID NO: 197)
LWWVNNQSLPVSP,
(SEQ ID NO: 198)
LWWVNNQSLPVSP,
(SEQ ID NO: 199)
EIIYPNASLLIQN,
(SEQ ID NO: 200)
NSIVKSITVSASG,
(SEQ ID NO: 201)
KTWGQYWQV,
(SEQ ID NO: 202)
(A)MLGTHTMEV,
(SEQ ID NO: 203)
ITDQVPFSV,
(SEQ ID NO: 204)
YLEPGPVTA,
(SEQ ID NO: 205)
LLDGTATLRL,
(SEQ ID NO: 206)
VLYRYGSFSV,
(SEQ ID NO: 207)
SLADTNSLAV,
(SEQ ID NO: 208)
RLMKQDFSV,
(SEQ ID NO: 209)
RLPRIFCSC,
(SEQ ID NO: 210)
LIYRRRLMK,
(SEQ ID NO: 211)
ALLAVGATK,
(SEQ ID NO: 212)
IALNFPGSQK,
(SEQ ID NO: 213)
ALNFPGSQK,
(SEQ ID NO: 214)
ALNFPGSQK,
(SEQ ID NO: 215)
VYFFLPDHL,
(SEQ ID NO: 216)
RTKQLYPEW,
(SEQ ID NO: 217)
HTMEVTVYHR,
(SEQ ID NO: 218)
SSPGCQPPA,
(SEQ ID NO: 219)
VPLDCVLYRY,
(SEQ ID NO: 220)
LPHSSSHWL,
(SEQ ID NO: 221)
SNDGPTLI,
(SEQ ID NO: 222)
GRAMLGTHTMEVTVY,
(SEQ ID NO: 223)
WNRQLYPEWTEAQRLD,
(SEQ ID NO: 224)
TTEWVETTARELPIPEPE,
(SEQ ID NO: 225)
TGRAMLGTHTMEVTVYH,
(SEQ ID NO: 226)
GRAMLGTHTMEVTVY,
(SEQ ID NO: 227)
SVSESDTIRSISIAS,
(SEQ ID NO: 228)
LLANGRMPTVLQCVN,
(SEQ ID NO: 229)
RMPTVLQCVNVSVVS,
(SEQ ID NO: 230)
PLLENVISK,
(SEQ ID NO: 231)
(E)AAGIGILTV,
(SEQ ID NO: 232)
ILTVILGVL,
(SEQ ID NO: 234)
EAAGIGILTV,
(SEQ ID NO: 235)
AEEAAGIGIL(T),
(SEQ ID NO: 236)
RNGYRALMDKS,
(SEQ ID NO: 237)
EEAAGIGILTVI,
(SEQ ID NO: 238)
AAGIGILTVILGVL,
(SEQ ID NO: 239)
APPAYEKLpSAEQ,
(SEQ ID NO: 240)
EEAAGIGILTVI,
(SEQ ID NO: 241)
RNGYRALMDKSLHVGTQCALTRR,
(SEQ ID NO: 242)
MPREDAHFIYGYPKKGHGHS,
(SEQ ID NO: 243)
KNCEPVVPNAPPAYEKLSAE,
(SEQ ID NO: 244)
SLSKILDTV,
(SEQ ID NO: 245)
LYSACFWWL,
(SEQ ID NO: 246)
FLTPKKLQCV,
(SEQ ID NO: 247)
VISNDVCAQV,
(SEQ ID NO: 248)
VLHWDPETV,
(SEQ ID NO: 249)
MSLQRQFLR,
(SEQ ID NO: 250)
ISPNSVFSQWRVVCDSLEDYD,
(SEQ ID NO: 251)
SLPYWNFATG,
(SEQ ID NO: 252)
SVYDFFVWL,
(SEQ ID NO: 253)
TLDSQVMSL,
(SEQ ID NO: 254)
LLGPGRPYR,
(SEQ ID NO: 255)
LLGPGRPYR,
(SEQ ID NO: 256)
ANDPIFVVL,
(SEQ ID NO: 257)
QCTEVRADTRPWSGP,
(SEQ ID NO: 258)
ALPYWNFATG,
(SEQ ID NO: 259)
KCDICTDEY,
(SEQ ID NO: 260)
SSDYVIPIGTY,
(SEQ ID NO: 261)
MLLAVLYCL,
(SEQ ID NO: 262)
CLLWSFQTSA,
(SEQ ID NO: 263)
YMDGTMSQV,
(SEQ ID NO: 264)
AFLPWHRLF,
(SEQ ID NO: 265)
QCSGNFMGF,
(SEQ ID NO: 266)
TPRLPSSADVEF,
(SEQ ID NO: 267)
LPSSADVEF,
(SEQ ID NO: 268)
LHHAFVDSIF,
(SEQ ID NO: 269)
SEIWRDIDF,
(SEQ ID NO: 270)
QNILLSNAPLGPQFP,
(SEQ ID NO: 271)
SYLQDSDPDSFQD
or
(SEQ ID NO: 272)
FLLHHAFVDSIFEQWLQRHRP.

Exemplary antigens overexpressed in tumour are:

(SEQ ID NO: 273)
SVASTITGV,
(SEQ ID NO: 274)
RSDSGQQARY,
(SEQ ID NO: 275)
LLYKLADLI,
(SEQ ID NO: 276)
YLNDHLEPWI,
(SEQ ID NO: 277)
CQWGRLWQL,
(SEQ ID NO: 278)
VLLQAGSLHA,
(SEQ ID NO: 279)
KVHPVIWSL,
(SEQ ID NO: 280)
LMLQNALTTM,
(SEQ ID NO: 281)
LLGATCMFV,
(SEQ ID NO: 282)
NPPSMVAAGSVVAAV,
(SEQ ID NO: 283)
ALGGHPLLGV,
(SEQ ID NO: 284)
TMNGSKSPV,
(SEQ ID NO: 285)
RYQLDPKFI,
(SEQ ID NO: 286)
DVTFNIICKKCG,
(SEQ ID NO: 287)
FMVEDETVL,
(SEQ ID NO: 288)
FINDEIFVEL,
(SEQ ID NO: 289)
KYDCFLHPF,
(SEQ ID NO: 290)
KYVGIEREM,
(SEQ ID NO: 291)
NTYASPRFK,
(SEQ ID NO: 292)
HLSTAFARV,
(SEQ ID NO: 293)
KIFGSLAFL,
(SEQ ID NO: 294)
IISAVVGIL,
(SEQ ID NO: 295)
ALCRWGLLL,
(SEQ ID NO: 296)
ILHNGAYSL,
(SEQ ID NO: 297)
RLLQETELV,
(SEQ ID NO: 298)
VVLGVVFGI,
(SEQ ID NO: 299)
YMIMVKCWMI,
(SEQ ID NO: 300)
HLYQGCQVV,
(SEQ ID NO: 301)
YLVPQQGFFC,
(SEQ ID NO: 302)
PLQPEQLQV,
(SEQ ID NO: 303)
TLEEITGYL,
(SEQ ID NO: 304)
ALIHHNTHL,
(SEQ ID NO: 305)
PLTSIISAV,
(SEQ ID NO: 306)
VLRENTSPK,
(SEQ ID NO: 307)
TYLPTNASL,
(SEQ ID NO: 308)
ALLEIASCL,
(SEQ ID NO: 309)
WLPFGFILI,
(SEQ ID NO: 310)
SPRWWPTCL,
(SEQ ID NO: 311)
GVALQTMKQ,
(SEQ ID NO: 312)
FMNKFIYEI,
(SEQ ID NO: 313)
QLAVSVILRV,
(SEQ ID NO: 314)
LPAVVGLSPGEQEY,
(SEQ ID NO: 315)
VGQDVSVLFRVTGALQ,
(SEQ ID NO: 316)
VLFYLGQY,
(SEQ ID NO: 317)
TLNDECWPA,
(SEQ ID NO: 318)
GLPPDVQRV,
(SEQ ID NO: 319)
SLFPNSPKWTSK,
(SEQ ID NO: 320)
STAPPVHNV,
(SEQ ID NO: 321)
LLLLTVLTV,
(SEQ ID NO: 322)
PGSTAPPAHGVT,
(SEQ ID NO: 323)
LLGRNSFEV,
(SEQ ID NO: 324)
RMPEAAPPV,
(SEQ ID NO: 325)
SQKTYQGSY,
(SEQ ID NO: 326)
PGTRVRAMAIYKQ,
(SEQ ID NO: 327)
HLIRVEGNLRVE,
(SEQ ID NO: 328)
TLPGYPPHV,
(SEQ ID NO: 329)
CTACRWKKACQR,
(SEQ ID NO: 330)
VLDGLDVLL,
(SEQ ID NO: 331)
SLYSFPEPEA,
(SEQ ID NO: 332)
ALYVDSLFFL,
(SEQ ID NO: 333)
SLLQHLIGL,
(SEQ ID NO: 334)
LYVDSLFFL,
(SEQ ID NO: 335)
NYARTEDFF,
(SEQ ID NO: 336)
LKLSGVVRL,
(SEQ ID NO: 337)
PLPPARNGGL,
(SEQ ID NO: 338)
SPSSNRIRNT,
(SEQ ID NO: 339)
LAALPHSCL,
(SEQ ID NO: 340)
GLASFKSFLK,
(SEQ ID NO: 341)
RAGLQVRKNK,
(SEQ ID NO: 342)
ALWPWLLMA(T),
(SEQ ID NO: 343)
NSQPVWLCL,
(SEQ ID NO: 344)
LPRWPPPQL,
(SEQ ID NO: 345)
KMDAEHPEL,
(SEQ ID NO: 346)
AWISKPPGV,
(SEQ ID NO: 347)
SAWISKPPGV,
(SEQ ID NO: 348)
MIAVFLPIV,
(SEQ ID NO: 349)
HQQYFYKIPILVINK,
(SEQ ID NO: 350)
ELTLGEFLKL,
(SEQ ID NO: 351)
ILAKFLHWL,
(SEQ ID NO: 352)
RLVDDFLLV,
(SEQ ID NO: 353)
RPGLLGASVLGLDDI,
(SEQ ID NO: 354)
LTDLQPYMRQFVAHL,
(SEQ ID NO: 355)
SRFGGAVVR,
(SEQ ID NO: 356)
TSEKRPFMCAY,
(SEQ ID NO: 357)
CMTWNQMNL,
(SEQ ID NO: 358)
LSHLQMHSRKH
or
(SEQ ID NO: 359)
KRYFKLSHLQMHSRKH,

A preferred tumour antigen is a lysate or homogenate of the tumour to be treated, more preferably a fraction thereof soluble in aqueous media, e.g. soluble in physiological saline and/or in medium containing DCs. Tumour lysate or tumour homogenate can e.g. originate from a tumour biopsy or from a surgery of the later recipient of the pharmaceutical combination of compounds.

It has been found that the administration of the first composition and subsequent administration of the second composition results in the generation of target antigen-specific activated T-cells. In comparison to other compositions, the combination of the invention results also in a higher proportion of target antigen-specific activated T-cells of all activated T-cells. The number and proportion of target antigen-specific T-cells was determined by staining a peripheral blood sample for IFN gamma following in vitro contacting with the target antigen using brefeldin A (GolgiPlug, available from Becton Dickinson) and immuno staining with a labelled anti-IFN gamma antibody and detection in flow cytometry. The number of all activated T-cells was determined by staining a peripheral blood sample with anti-CD11a antibody and detection in flow cytometry. Accordingly, activated CD8+ T-cells are CD11a^{hi}, tetramer-positive for the antigen and/or are IFNγ positive. The effect of inducing a target antigen-specific T-cell response, wherein the target antigen preferably is a tumour antigen, within e.g. 10 to 14 days is currently believed to be based on the combination of the first composition providing APC specifically primed for the target antigen, which APC preferably are DC, with the second composition providing a specific boost for the T-cells that were stimulated by the DCs loaded with target antigen by the combination of the malignant antigen with the co-stimulatory antibody for T-cells, preferably in combination with the non-specific agonist for TLR3, for TLR7, for TLR4 and/or for TLR9. Accordingly, the first composition and its administration can also be termed priming, and the second composition and its administration can be termed boosting.

In the embodiment in which the APC loaded with target antigen contained in the first composition are replaced with target antigen coupled to an antibody specific for the APC, especially coupled to an antibody specific for a DC surface receptor, e.g. anti-DEC205 antibody or anti-DCIR antibody (anti-dendritic cell immunoreceptor), the target antigen-loaded APC are generated vivo by administration of the first composition.

It is currently assumed that the second composition due to its content of the target antigen preferentially boosts malignant antigen-specific T-cells from the pool of primed T-cells present in the recipient. This is an advantage over the use of co-stimulating antibody and/or of a non-specific TLR3 agonist alone or in combination as these can be expected to activate all primed T-cells irrespective of their antigen specificity, resulting in a proportionate boost of target antigen-specific T-cells only. Accordingly, the second composition is also assumed to have the advantage of boosting non-target specific T-cells to a lesser extent than the use of co-stimulating antibody and/or of a non-specific TLR3 agonist alone.

Advantageously, the administration of the second composition following the administration of the first composition is with a temporal delay of approx. 1 to 7 days.

The pharmaceutical combination of compounds has the advantage of raising in a recipient an effective T-cell immunity also against an intracellular tumour antigen.

For the purpose of the invention, an antibody, e.g. an antibody specific for a DC surface receptor, a co-stimulatory agonistic antibody for CD8+ T-cells, can be a natural antibody, e.g. IgG, or a synthetic peptide having a paratope of the specificity, e.g. a diabody, minibody etc.

In the following examples and comparative examples, mice were used for representing a human recipient. Mice were divided into groups of 5 mice (strain C57 Bl/6) each. The animals were housed under standard conditions with feed and water ad libitum. Mice were subjected to different prime-boost regimens. Administration of first composition (priming) and of second composition (boosting) was by intravenous (iv) injection. In the figures, the co-stimulatory antibody is designated by its target, e.g. in the figures anti-CD40 antibody is designated as CD40.

As an example for a malignant antigen, a mouse antigen isolated from HCC tumour, Ndufs1 having amino acid AAVSNMVQKI (SEQ ID NO: 360) was used. Ndufs1 is a model antigen for a homologous tumour antigen. Ndufs1 was prepared by chemical peptide synthesis. The compositions comprised the constituents of the compositions in aqueous medium, preferably in physiological saline.

### Example 1: Immunization with Different First Compositions, Followed by Different Second Compositions

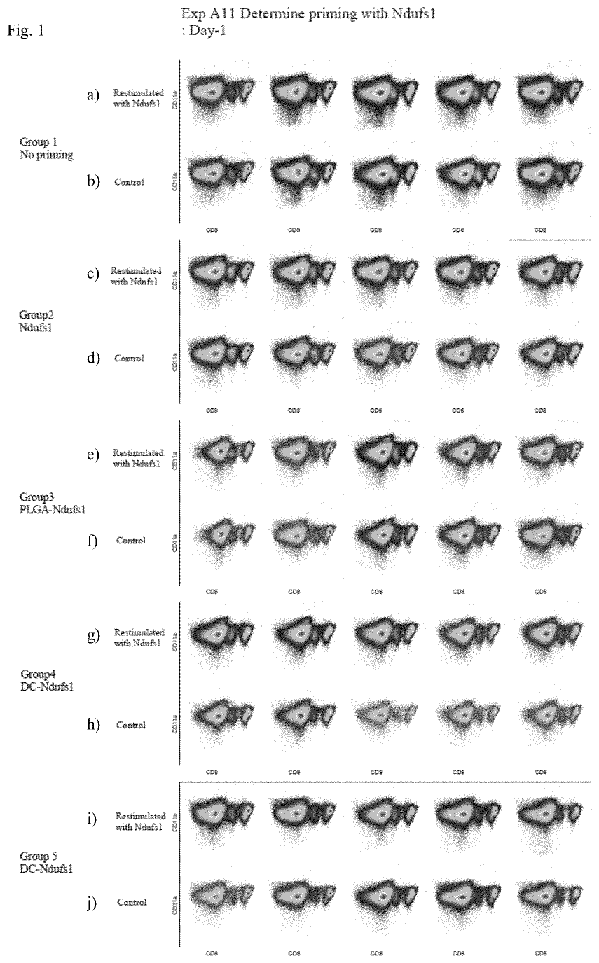

For priming, on day -7 mice received as a first composition either physiological saline (group 1), 100 μg soluble Ndufs1 peptide (group 2), 100 μg Ndufs1 peptide conjugated to 1 mg PLGA microspheres of 2 μm mean diameter (group 3), or 10⁶ dendritic cells that were in vitro coated with 10 μg Ndufs1 peptide (groups 4 and 5) intravenously. 7 days later (day 0), mice received boosting by intravenous administration of a combination of 100 μg Ndufs1 peptide, 100 μg of agonistic anti-CD40 antibody (clone 1C10) and 200 μg of Poly(I:C) (groups 1 to 4), or again 10⁶ dendritic cells that were in vitro coated with 10 μg Ndufs1 peptide (group 5) as the second composition. After the administration of the second composition, mice were bled from the mandibular vein on the days indicated below. After red cell lysis, peripheral blood mononuclear cells were stained with the following labelled antibodies: anti-IFN gamma antibody-APC (clone XMG1.2, eBioscience), anti-CD8 antibody-FITC (53-6.7, eBioscience and Becton Dickinson Biosciences), anti-CD11a antibody-PE (M17/4, eBioscience). The results of flow cytometry using a model Canto II flow cytometer (Becton Dickinson Biosciences) are shown in FIG. 1.

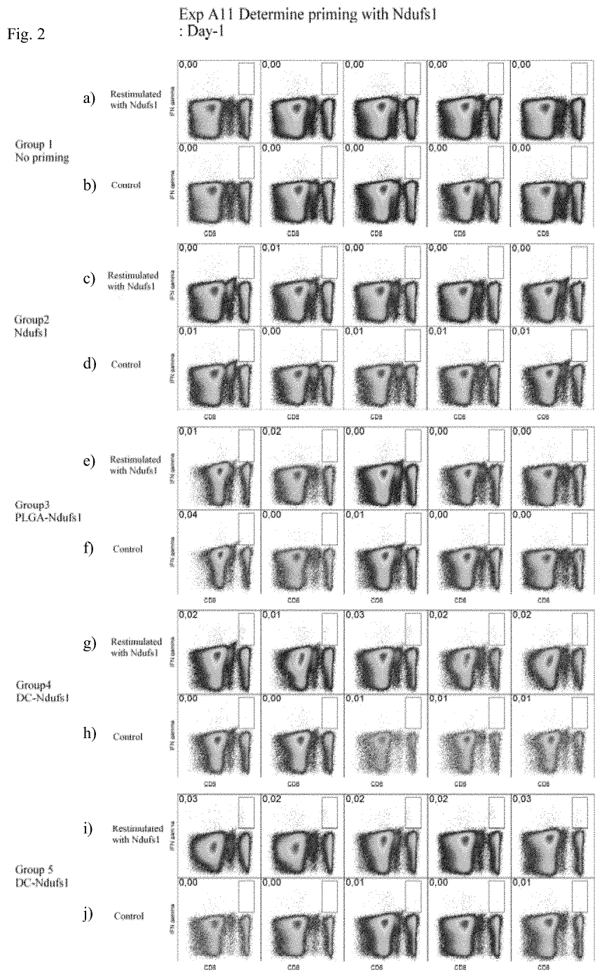

The following table summarizes first compositions followed by administration of the second compositions:

| Group (Gr.) | results in | priming | boosting |
|---|---|---|---|
| 1 | FIG. 1a), 1b) | physiological saline | Ndufs1 + Poly (I:C) + |
| | FIG. 2a), 2b) | (no priming) | anti-CD40 |
| 2 | FIG. 1c), d) | Ndufs1 only | Ndufs1 + Poly (I:C) + |
| | FIG. 2c), d) | (Ndufs1) | anti-CD40 |
| 3 | Fig. 1e), f) | PLGA-Ndufs1 | Ndufs1 + Poly (I:C) + |
| | FIG. 2e), f) | | anti-CD40 |
| 4 | FIG. 1g), h) | DC-Ndufs1 | Ndufs1 + Poly (I:C) + |
| | FIG. 2g), h) | | anti-CD40 |
| 5 | FIG. 1i), j) | DC-Ndufs1 | DC-Ndufs1 |
| | FIG. 2i), j) | | |

The 5 animals of each group were treated identically.

PLGA-Ndufs1 designates microspheres of poly(lactic-co-glycolic) acid comprising the model antigen Ndufs1. DC-Ndufs1 designates dendritic cells (DCs) isolated from the spleen of a mouse of the same strain without administration of the antigen Ndufs1, which DCs were incubated in RPMI culture medium and loaded with the antigen by adding Ndufs1 to a concentration of approx. 2 μg/ml medium.

FIGS. 1-3 refer to analyses at day −1, i.e. 6 days following administration of the respective first composition and 1 day prior to administration of the respective second composition.

FIG. 1 shows the results of analysis for CD11a, indicating the activated T-cells of the total T-cells (CD8), with addition of antigen Ndufs1 (Figs. a), c), e), g) and i)) in one sample of each animal and without added antigen (Control) for each sample of each animal (Figs. b), d), h) and j)).

The analysis for target antigen-specific T-cells was by measuring IFN gamma following re-stimulation with antigen Ndufs1 (FIGS. 2 a), c), e), g) and i)) in a sample of each animal in comparison to the samples from the same animals without added antigen (FIGS. 2 b), d), f), h) and j)). For measurement of IFN gamma produced by each T-cell (CD8), secretion of IFN gamma was hindered by addition of brefeldin A (GolgiPlug, available from Becton Dickinson), followed by staining using a labelled anti-IFN gamma antibody and measurement by flow cytometry.

The flow cytometry analyses after administration of the first compositions only (P, priming) are summarized in FIG. 3, showing that a first composition consisting of DCs in vitro loaded the model tumour antigen Ndufs in groups 4 and 5 (DC-Ndufs1) resulted in approx. 0.02% specific CD8+ T-cells in peripheral blood lymphocytes (PBL), whereas priming with PLGA microspheres with the antigen, group 3 (PLGA-Ndufs1) or antigen alone in group 2 (Ndufs1 ) resulted in numbers of antigen-specific CD8+ T-cells in PBL close to background obtained without antigen (Gr. 1, -).

Following administration of the second compositions at day 0 to the same animals, samples were taken 7 days later (Day 7).

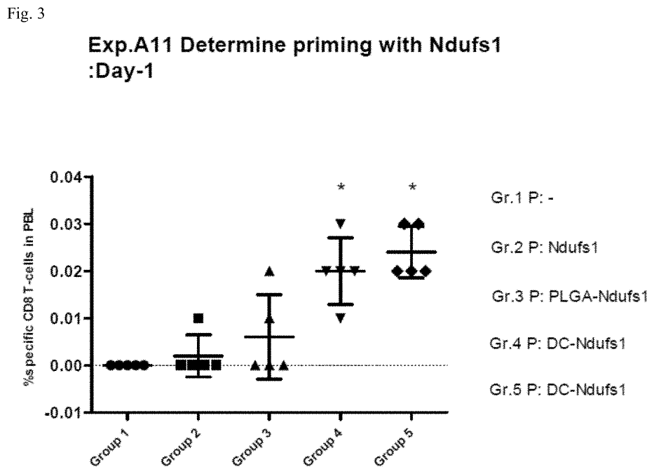

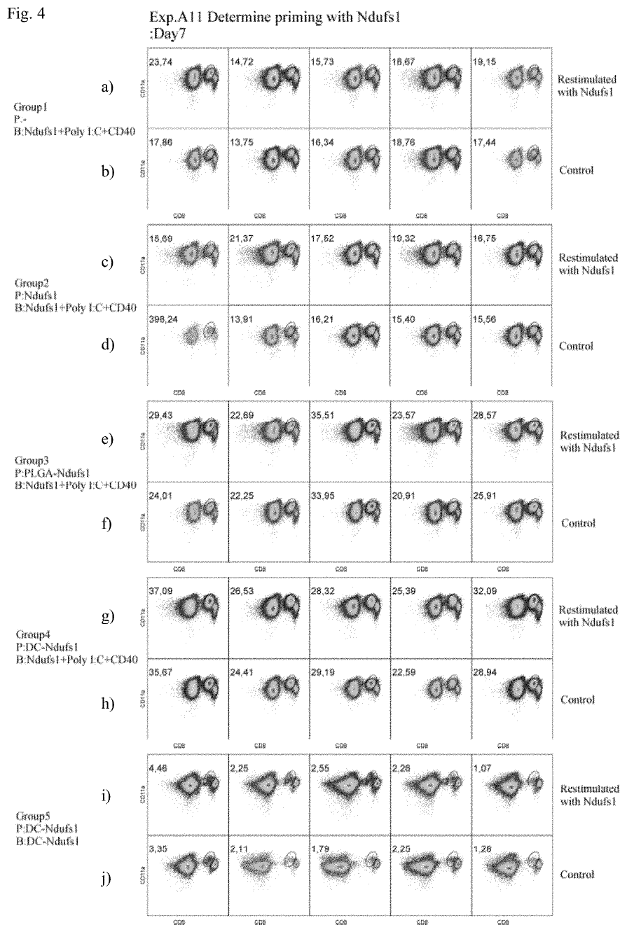

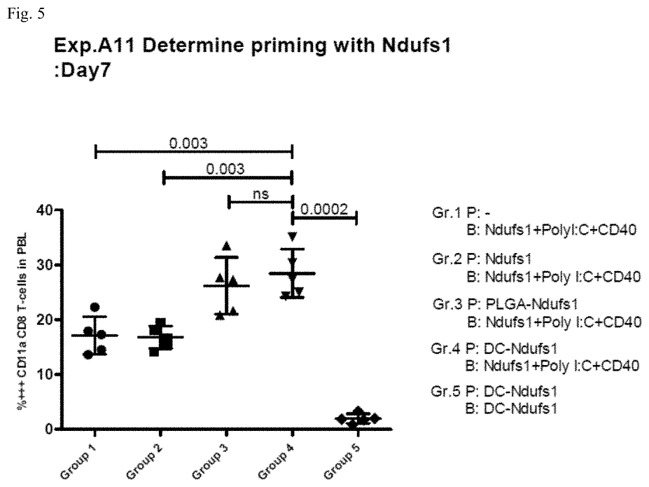

The flow cytometry results from FIG. 4 for staining with anti-CD11a antibody (CD11a) and anti-CD8 antibody (CD8), using the encircled areas, are summarized in FIG. 5, showing that CD11a high positive T-cells (+++CD11a), which are activated T-cells, are generated to approx. 27-28% by the first compositions of PLGA microspheres comprising the antigen or DC loaded with antigen, followed by administration of the second composition comprising the antigen, the TLR3 agonist Poly(I:C) and the co-stimulating antibody anti-CD40 antibody. In contrast, the priming with antigen-primed DC followed by boosting with antigen-primed DC gave the lowest number of activated CD8+ T-cells.

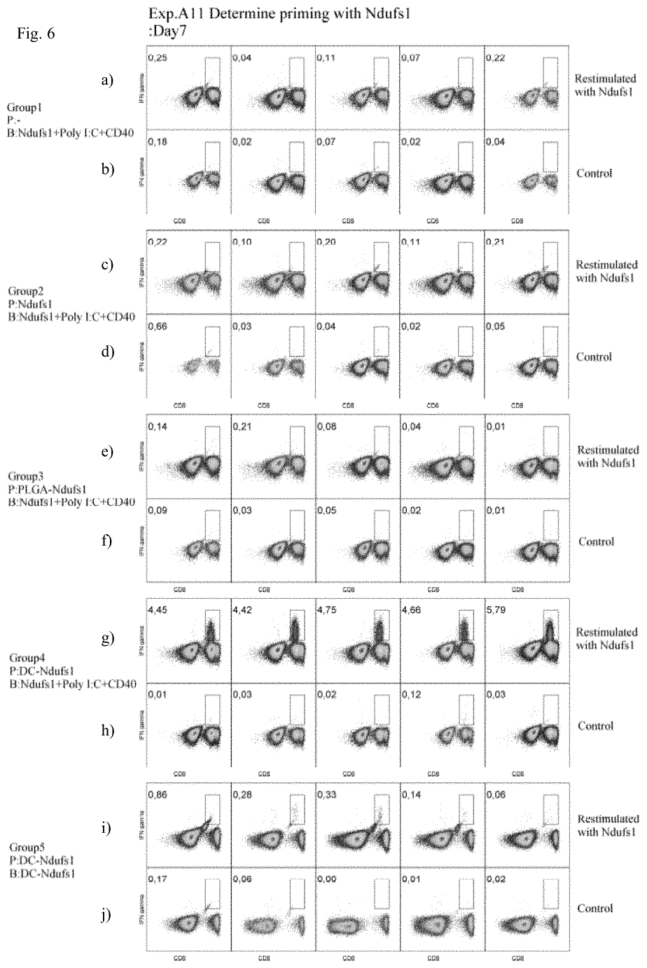

FIG. 6 shows the flow cytometry results for staining with anti-IFN gamma (IFN gamma) and anti-CD8+ (CD8), following re-stimulation with antigen Ndufs1 (FIGS. 6 a), c), e), g) and i)) and without added antigen (FIGS. 6 b), d), f), h) and j)). It is clearly seen that the priming with a first composition comprising DC loaded with antigen followed by boosting with a second composition comprising the antigen in combination with the TLR3 agonist and with the co-stimulating antibody according to the invention yields the most effective generation of a proportion of antigen-specific CD8+ T-cells (insets in FIG. 6g)) in activated T-cells, which are highly CD11a positive CD8+ T-cells (+++CD11a CD8 T-cells), approx. 16% (Group 4). This proportion is significantly higher than that obtained for both priming and boosting by antigen-loaded DC (Group 5).

Interestingly, FIG. 6 e) shows that priming by a first composition of PLGA microspheres coated with the Ndufs1 antigen does not yield a detectable level of antigen-specific CD8+ T-cells upon restimulation with Ndufs1 plus TLR3 agonist and the co-stimulatory antibody anti-CD40 (CD40). Currently, this absence of a boosting effect by the second composition is assumed to be caused by the antigen being a tumour-specific antigen and/or when the first composition contains the antigen bound to PLGA microspheres.

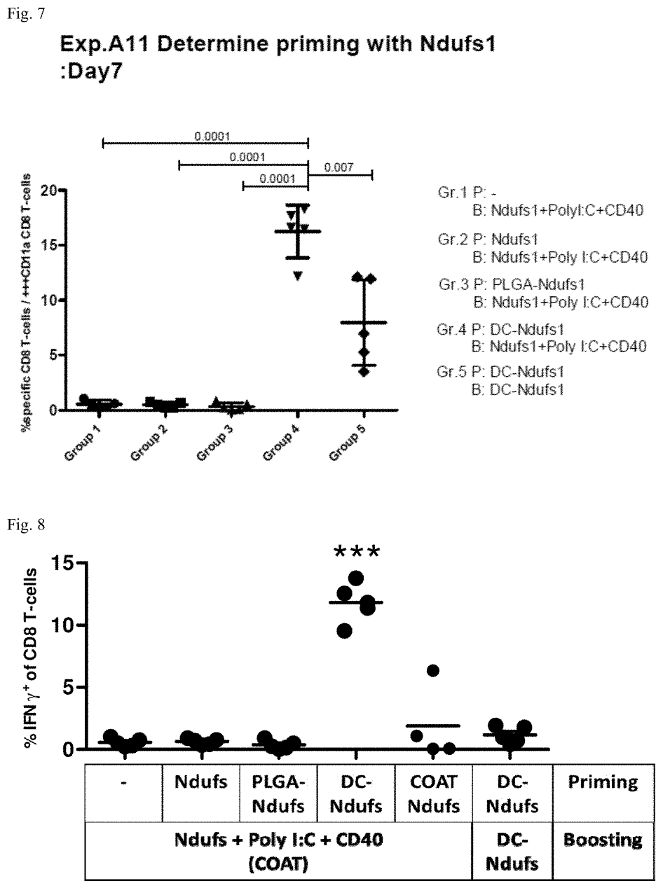

The analytical data for the different first and second compositions for the experimental animals of each group are summarized in FIG. 7. The data show that the combination of compositions according to the invention, priming by antigen-loaded DC followed by boosting with the antigen in combination with an co-stimulatory antibody and, optionally a TLR agonist, results in the highest activation of antigen-specific CD8+ T-cells (Gr. 4), whereas priming with antigen-coated PLGA carrier did not result in a relevant antigen-specific CD830 T-cell generation when using the same boost (Gr. 3).

Further, the proportion of IFNγ-positive cells in CD8+ T-cells was determined for different first and second compositions administered: no priming (-), tumour antigen only (Ndufs), PLGA coated with antigen (PLGA-Ndufs), DC coated with tumour antigen (DC Ndufs) Ndufs1 plus Poly(I:C) plus anti-CD40 antibody (COAT Ndufs), followed as indicated in FIG. 8 by the second composition Ndufs1 plus Poly(I:C) plus anti-CD40 antibody (Ndufs+PolyI:C +CD40+COAT). As a further comparison, both the first and second composition were DC coated with Ndufs1 as the antigen (DC-Ndufs). The data of FIG. 8 show that only the combination of the first and second compositions according to the invention result in an effective generation of antigen-specific IFNγ-positive CD8+ T-cells. Priming by PLGA coated with antigen (PLGA-Ndufs) gave a highly significant lower proportional response by IFNγ positive CD8+ T-cells than priming with DC coated with the same tumour antigen (DC-Ndufs), when both were followed by a boost of the tumour antigen Ndufs1, TLR agonist Poly(I:C) and co-stimulatory antibody anti-CD440 (Ndufs+Poly I:C+CD40) (COAT).

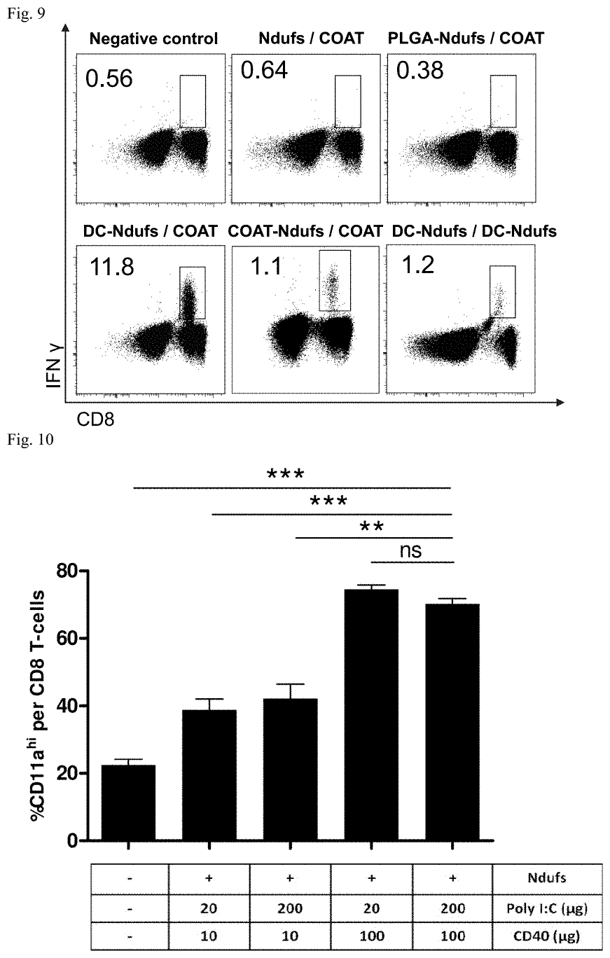

FIG. 9 (all axis same scale, Y-axis IFNγ, X-axis CD8+) shows FACS results for individual experimental mice analysed in FIG. 8 which have a mean immune response in the respective group as indicated by the inset number showing the relative proportion of IFNγ positive CD8+ T-cells, e.g. for the compositions of the invention in FIG. 9 d) a value of 11.8% for the first composition of DC-Ndufs and the second composition of Ndufs1, stimulatory antibody and TLR agonist (COAT).The box indicates tumour-antigen specific CD8+ T-cells. The negative control in FIG. 9a) consisting of priming by antigen Ndufs1 (Ndufs) and boosting by COAT gave a marginal response of IFNγpositive CD8+ T-cells (0.56), FIG. 9b) for priming by antigen alone (Ndufs) and boosting by COAT gave a response of 0.64, and PLGA coated with antigen gave an even lower specific response. The comparative priming and boosting by DC coated with antigen (DC-Ndufs) of FIG. 9f) gave a response a little higher than the other comparative compositions.

Also the results of FIG. 9 show that essentially only the combination of compositions according to the invention results in an effective generation of tumour-antigen specific CD8+ T-cells.

### Example 2: Generation of Antigen-Specific CD8+ T-Cell Response Against Tumour-Antigen

Follwing administration of the first composition at day −7, consisting of 10⁶ dendritic cells that were in vitro coated with 10 μg Ndufs1 peptide in a vehicle, mice were administered with second compositions of the antigen100 μg Ndufs1 and varying amounts of co-stimulatory antibody, exemplified by anti-CD40, and varying amounts of TLR agonist poly(I:C). The results are shown in FIG. 10 for no boost by a second composition (left hand col., -Ndufs, -Poly I:C, -antibody (CD40)), and with the amounts indicated.

The results show that the co-stimulatory antibody of the second composition has a significant effect on the generation of the T-cell response, whereas the TLR agonist supports the effect the second composition, e.g. a comparison of 10 μg anti-CD40 with 20 μg or 200 μg Poly(I:C) shows raising similar proportions of CD11a^{hi} CD8+ T-cells in total CD8+ T-cells; the same can be seen for 100 μg anti-CD40, drastically raising the proportion of CD11a^{hi} CD8+ T-cells compared to 10 μg anti-CD40, whereas 20 μg or 200 μg Poly(I:C) have a less important impact.

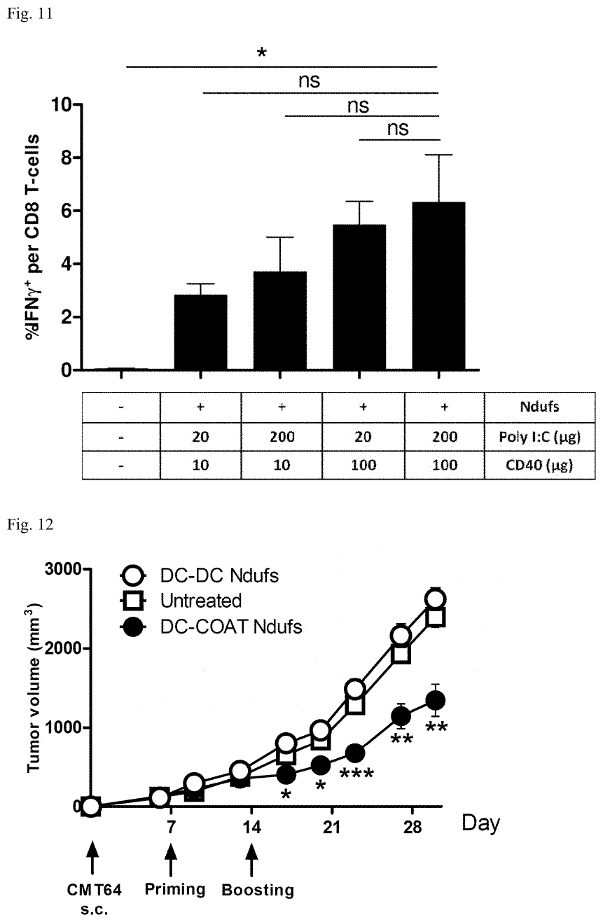

The results of the analysis of IFNγ-positive CD8+ T-cells in relation to total CD8+ T-cells are shown in FIG. 11. The data show that the combination of a co-stimulatory antibody and a TLR agonist in the second composition improves the proportion of activated tumour-specific CD8+ T-cells.

### Example 3: In Vivo Treatment of Tumour

As an example for a tumour, mice were subcutaneously injected with 10⁷ CMT 64 cells (mouse lung carcinoma) to generate solid subcutaneous tumours seven days prior to the beginning of the immunisations.

Mice were administered with 10⁶ dendritic cells that were in vitro coated with 10 μg Ndufs1 peptide intravenously on day −7. 7 days later (day 0), mice received the same composition again (DC-DC Ndufs),

or according to the invention with 10⁶ dendritic cells that were in vitro coated with 10 μg Ndufs1 peptide intravenously on day −7. 7 days later (day 0), mice received boosting by intravenous administration of a combination of 100 μg Ndufs1 peptide, 100 μg of agonistic anti-CD40 antibody (clone 1C10) and 200 μg of Poly(I:C) (DC-COAT Ndufs), or mice were left without treatment as a negative control (Untreated).

The results are shown in FIG. 12, demonstrating that the treatment by DC coated with antigen followed by the boosting second composition comprising the tumour antigen, a co-stimulatory antibody and a TLR agonist resulted in a significantly reduced growth of the tumour (CMT 64), whereas the administration of DC coated with the antigen as both the first and the second composition did not result in a significant difference of tumour growth, possibly in added tumour growth.

### Example 4: Immunization With First Compositions Containing DC Primed with SIINFEKL, Followed by Different Second Compositions

For priming, on day −7 mice received as a first composition 10⁶ dendritic cells that were in vitro coated with 10 μg SIINFEKL peptide, the antigenic epitope of hen ovalbumin (OVA), intravenously and at day 0 were challenged with a second composition by intravenous administration of a combination of 100 μg SIINFEKL peptide, 100 μg of agonistic antibody, and 200 μg of Poly(I:C) for the co-stimulatory antibodies indicated in FIGS. 8 to 10: anti-CD40 (CD40) (clone 1C10), anti-CD134 (CD134) (also termed OX40), anti-CD137 2EI (CD137 2EI), anti-CD137 3H3 (CD137 3H3), anti-CD278 (CD278), corresponding to ICOS.

As a negative control, the co-stimulatory antibody was replaced by rat IgG2 (RatIgG2) in the second composition. As a positive control, mice on day −7 received Listerium monocytogenes expressing ovalbumin (LM-OVA) followed again by LM-OVA at day 0 as the second composition. As a further negative control, mice were not treated at day −7 nor at day 0 (naiv).

7 days after the administration of the second composition, mice were bled from the mandibular vein. After red cell lysis, peripheral blood mononuclear cells were stained with the following labelled antibodies: anti-CD8 antibody-FITC (53-6.7, eBioscience and Becton Dickinson Biosciences), anti-CD11a antibody-PE (M17/4, eBioscience), TET+ was detected by SIINFEKL-specific tetramers in order to identify antigen-specific activated T-cells.

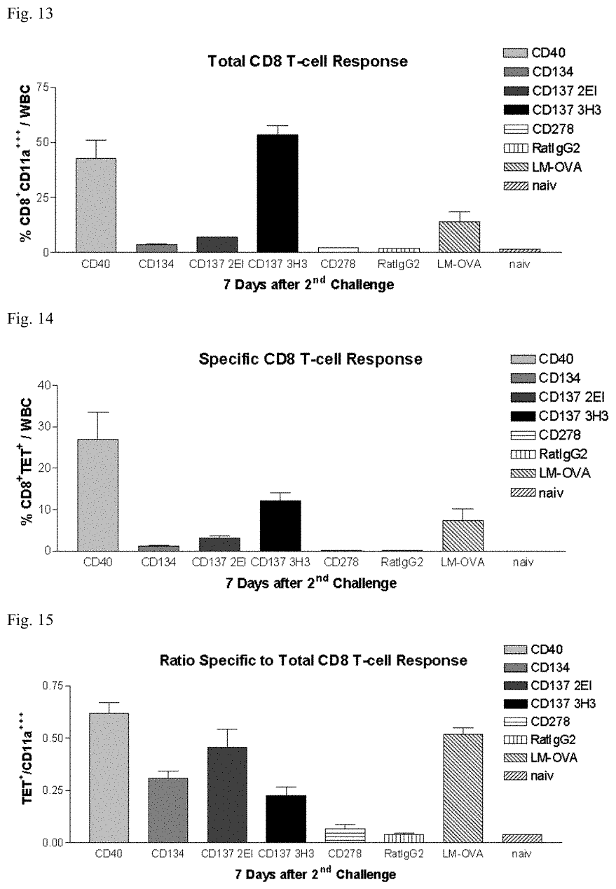

The proportion of antigen-specific activated T-cells (CD8+ and CD11a+++ T-cells) of white blood cells (WBC) is shown in FIG. 13 at day 7 (7 Days after 2^{nd} challenge) following the administration of the second composition shows high proportions of activated T-cells for the agonistic antibody anti-CD40 (CD40), and for anti-CD137 3H3, which are higher than the proportion obtained by the positive control LM-OVA.

FIG. 14 shows that the antigen-specific CD8 T-cell response to the heterologous antigen SIINFEKL in relation to total white blood cells (WBC) was most intense for boosting with anti-CD40 or anti-CD137 3H3 as the co-stimulatory antibody, and also higher than the positive control LM-OVA.

FIG. 15 shows that proportion of the antigen-specific CD8 T-cell response in total activated CD8+T-cells for the co-stimulatory antibodies anti-CD40 (CD40), anti-CD134 (CD134), anti-CD137 2EI (CD137 2EI), anti-CD137 3H3 (CD137 3H3) and anti-CD278 (CD278) (ICOS) was higher than the negative control (naiv) and negative control RatIgG2 for all of the co-stimulatory antibodies tested. Accordingly, these results show that a co-stimulatory antibody which according to the invention is directed against a surface receptor of T-cells and/or of DCs, in the second composition raises the antigen-specific CD8+ T-cell response.

In comparison to the negative control RatIgG, these data show that the presence of a co-stimulatory antibody has a high influence on the generation of the antigen-specific CD8+ T-cell response.

### Example 5: Activity of CD8+ T-Cell Immune Response

For comparing the effect of the combination of the first and second composition for activity against cells expressing a malignant antigen, SIINFEKL was used as the antigen according to the invention (DC COAT) in comparison to virulent Listerium monocytogenes, representing an intracellular bacterial antigen, and in comparison to LCM virus representing an intracellular viral antigen.

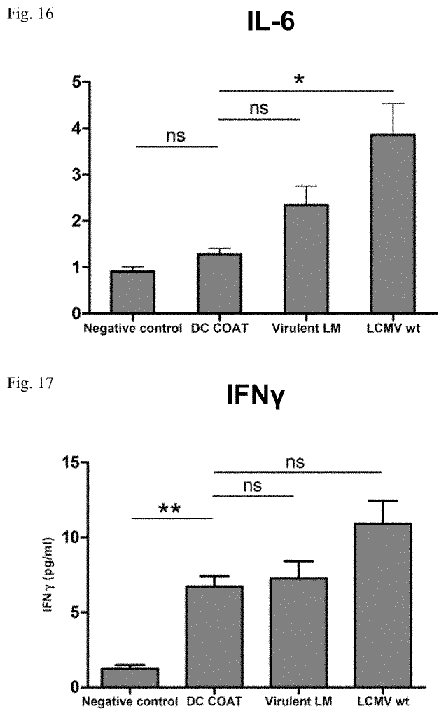

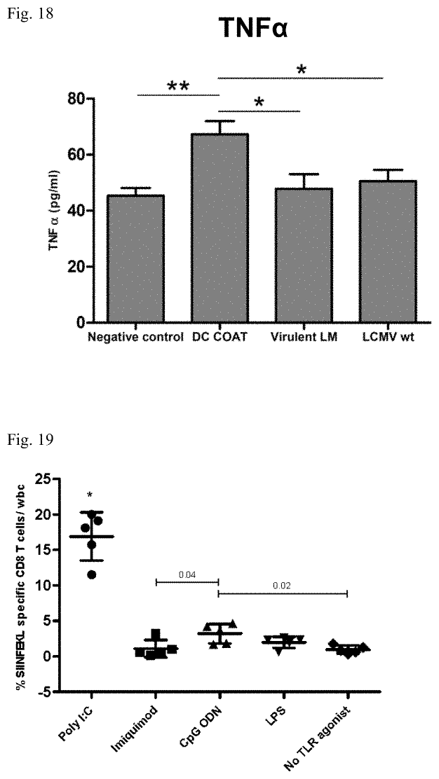

For priming according to the invention, on day −7 mice received as a first composition 10⁶ dendritic cells that were in vitro coated with 10 μg SIINFEKL peptide intravenously and at day 0 were challenged with a second composition by intravenous administration of a combination of 100 μg SIINFEKL peptide, 100 μg of anti-CD40 (CD40) (clone 1C10) as the co-stimulatory antibody, and 200 μg of Poly(I:C). This combination is designated as DC COAT in FIGS. 16-18.

Virulent Listerium monocytogenes (Virulent LM) was administered at a dose of 5×10⁴ cfu/mouse at day −7 at day 0.

LCM virus (LCMV, Armstrong wild-type strain) was administered at a concentration of 2×10⁵ at day 0.

As a negative control, mice were left without treatment or challenge.

For analysis, at day 7 following day 0, cytokines IL-6, IFNγ and TNFα were analysed from spleen lysate. The increased production of these cytokines as measured in spleen lysate indicates expansion of CD8+ T-cells and CD8+ T-cell activation.

Results are shown in FIGS. 16 to 18. The results show that the administration of the first and second compositions according to the invention (DC COAT) gave rise to CD8+ T-cell expansion as indicated by producing the significantly highest production of TNFa, indicating anti-tumour activity, in comparison to both intracellular pathogens represented by virulent LM and virulent LCM virus.

The results show that production of TNFα was best induced by the combination according to the invention when compared to the virulent bacterium or virus, demonstrating the high efficacy of the combination of the first and second compositions according to the invention for generating an antigen-specific CD8+ T-cell response.

The production of IFNγ by the CD8+ T-cells raised by DC COAT was not significantly higher than in the negative control and not significantly lower than that raised by virulent LM (FIG. 16), but significantly lower than that raised by LCM virus. This level of IL-6 shows the systemic inflammation induced by the boost (second composition) as a further proof of functional CD8+ T-cells induced by the vaccine combination of first and second compositions of the invention.

The production of IFNγ in CD8+ T-cells raised by the compositions of the invention are very significantly higher than that in the negative control and at a level comparable to that raised by LM or LCM virus. This level of IFNγ shows the active secretion of interferon γ by the CD8+ T-cells that were induced by the vaccine combination of first and second compositions. This active secretion of IFNγ is in contrast to anergic T-cells, which are IFNγ-secretion defective.

These results show a good anti-tumour function of the CD8+ T-cells raised by the compositions of the invention.

### Example 6: TLR Agonists in Second Composition

In order to assess the effect of a TLR agonist in the second composition, on day −7 mice received as a first composition 10⁶ dendritic cells that were in vitro coated with 10 μg SIINFEKL peptide intravenously and at day 0 were challenged with a second composition by intravenous administration of a combination of 100 μg SIINFEKL peptide, 100 μg of anti-CD40 (CD40) (clone 1C10) as the co-stimulatory antibody, and a TLR agonist. The results are shown in FIG. 19, showing that the highest proportion of antigen-specific CD8+ T-cells was obtained for the TLR agonist being 200 μg Poly(I:C) (Poly I:C), with the negative control (No TLR agonist) resulting in comparatively low proportions of antigen-specific CD8+ T-cells and the TLR7 agonist (Imiquimod), the TLR9 agonist CpG oligodesoxynucleotide (CpG ODN) or lipopolysaccharide of E. coli (LPS) giving significantly higher proportions of antigen-specific CD8+ T-cells.

These data show that the invention also in the absence of a TLR agonist in the second composition raises antigen-specific CD8+ T-cells, and that presence of a TLR agonist in the second composition is preferred, especially Poly(I:C) is the preferred TLR agonist.

Generally, it is preferred that the first composition is free from a co-stimulatory antibody and/or free from a TLR agonist.

1. A medical treatment method, the method comprising:
administering to a recipient a first composition comprising dendritic cells (DC) which are immunologically compatible with the recipient and which are associated with a target antigen; and
administering to the recipient a second composition comprising at least a portion of the target antigen in soluble form and a co-stimulatory antibody effective for activating T-cells and/or the dendritic cells (DC), wherein the second composition is administered at least 1 day subsequent to administration of the first composition.

2. The method according to claim 1, wherein the dendritic cells are autologous dendritic cells.

3. The method according to claim 1, wherein the dendritic cells (DC) are associated with the target antigen by being contacted with the target antigen or by being contacted with a nucleic acid sequence encoding the antigen.

4. The method according to claim 1, wherein the medical treatment is the treatment of tumour, of viral infections or of infections by intracellular bacteria.

5. The method according to claim 1, wherein the second composition further contains a TLR3 agonist, TLR7 agonist, TLR4 agonist, TLR9 agonist or combinations of at least two of these.

6. The method according to claim 1, wherein the co-stimulatory antibody effective for activating dendritic cells (DC) is selected from the group consisting of anti-CD137 antibody, an anti-CD40 antibody, an anti-OX40 antibody, anti-ICOS antibody, an anti-CD27 antibody, an anti-CD28 antibody, an anti-GITR antibody, an anti-human GITR/AITR antibody, an anti-HVEM antibody, an anti-TIM1 antibody, an anti-TIM3 antibody, and mixtures of at least two of these.

7. The method according to claim 6, wherein the TLR3 agonist is Poly(I:C) and/or PolyICLC or a homologue thereof.

8. The method according to claim 1, wherein the medical treatment is for raising in the recipient a cellular immune response specifically directed against cells of the recipient bearing the target antigen.

9. The method according to claim 1, wherein the first composition is free from an adjuvant.

10. The method according to claim 5, wherein the tumour is selected from the group comprising or consisting of hematological malignancies, Hodgkin and non-Hodgkin lymphomas, leukemias, especially acute lymphoblastic leukemia, acute myeloid leukemia, chronic lymphocytic leukemia, chronic myeloid leukemia, monocytic leukemia, myelomas, myeloproliferative diseases, myelodysplastic syndromes and solid cancers, especially originating from brain, head and neck, lung, pleura, heart, liver, kidney, colon, pancreas, stomach, gut, urinary tract, prostate, uterus, ovaries, breast, skin, testes, larynx and sarcoma.

11. The method according to claim 5, wherein the tumour antigen is selected from the group consisting of tumour antigens, tumour homogenate or tumour lysate.

12. The method according to claim 2, wherein the dendritic cells (DC) following in vitro contact with the target antigen by being contacted with the target antigen or by being contacted with a nucleic acid sequence encoding the antigen are separated from the medium containing the target antigen or nucleic acid sequence encoding the antigen and are expanded in number by cultivation in cell culture medium.

13. The method according to claim 1, wherein the medical treatment comprises the generation of CD8+ T-cells which are specific for the target antigen and/or the generation of CD4+ T-cells which are specific for the target antigen.

14. The method according to claim 1, wherein the medical treatment generates activated CD8+ T-cells having specificity for autologous cells comprising the antigen.

15. The method according to claim 1, wherein the second composition further comprises a TLR3 agonist that is Poly(I:C) and/or PolyICLC or a homologue thereof.


SEQUENCE LISTING (360 sequences)

SEQ ID NO: 1 - protein, 10 aa, Artificial Sequence
  tumour antigen for lung carcinoma
FIASNGVKLV

SEQ ID NO: 2 - protein, 15 aa, Artificial Sequence
  tumour antigen for melanoma
YSVYFNLPADTIYTN

SEQ ID NO: 3 - protein, 9 aa, Artificial Sequence
  tumour antigen for chronic myeloid leukemia
SSKALQRPV

SEQ ID NO: 4 - protein, 9 aa, Artificial Sequence
  tumour antigen for chronic myeloid leukemia
GFKQSSKAL

SEQ ID NO: 5 - protein, 17 aa, Artificial Sequence
  tumour antigen for chronic myeloid leukemia
ATGFKQSSKALQRPVAS

SEQ ID NO: 6 - protein, 17 aa, Artificial Sequence
  tumour antigen for chronic myeloid leukemia
ATGFKQSSKALQRPVAS

SEQ ID NO: 7 - protein, 29 aa, Artificial Sequence
  tumour antigen for melanoma
EDLTVKIGDFGLATEKSRWSGSHQFEQLS

SEQ ID NO: 8 - protein, 9 aa, Artificial Sequence
  tumour antigen for colorectal, gastgric , and endometrialt umour
FLIIWQNTM

SEQ ID NO: 9 - protein, 9 aa, Artificial Sequence
  tumour antigen for head and neck squamous cell carcinoma
FPSDSWCYF

SEQ ID NO: 10 - protein, 9 aa, Artificial Sequence
  tumour antigen for melanoma
SYLDSGIHF

SEQ ID NO: 11 - protein, 12 aa, Artificial Sequence
  tumour antigen for melanoma
FSWAMDLDPKGA

SEQ ID NO: 12 - protein, 10 aa, Artificial Sequence
  tumour antigen for melanoma
ACDPHSGHFV

SEQ ID NO: 13 - protein, 9 aa, Artificial Sequence
  tumour antigen for melanoma
AVCPWTWLR

SEQ ID NO: 14 - protein, 14 aa, Artificial Sequence
  tumour antigen for colorectal carcinoma
TLYQDDTLTLQAAG

SEQ ID NO: 15 - protein, 14 aa, Artificial Sequence
  tumour antigen for colorectal carcinoma
TLYQDDTLTLQAAG

SEQ ID NO: 16 - protein, 16 aa, Artificial Sequence
  tumour antigen for myeloid leukemia
TMKQICKKEIRRLHQY

SEQ ID NO: 17 - protein, 10 aa, Artificial Sequence
  tumour antigen for melanoma
KILDAVVAQK

SEQ ID NO: 18 - protein, 9 aa, Artificial Sequence
  tumour antigen for lung squamous CC
ETVSEQSNV

SEQ ID NO: 19 - protein, 9 aa, Artificial Sequence
  tumour antigen for acute lymphoblastic leukemia
RIAECILGM

SEQ ID NO: 20 - protein, 15 aa, Artificial Sequence
  tumour antigen for acute lymphoblastic leukemia
IGRIAECILGMNPSR

SEQ ID NO: 21 - protein, 15 aa, Artificial Sequence
  tumour antigen for acute lymphoblastic leukemia
IGRIAECILGMNPSR

SEQ ID NO: 22 - protein, 10 aa, Artificial Sequence
  tumour antigen for acute myelogenous leukemia
YVDFREYEYY

SEQ ID NO: 23 - protein, 14 aa, Artificial Sequence
  tumour antigen for melanoma
MIFEKHGFRRTTPP

SEQ ID NO: 24 - protein, 10 aa, Artificial Sequence
  tumour antigen for melanoma
TLDWLLQTPK

SEQ ID NO: 25 - protein, 9 aa, Artificial Sequence
  tumour antigen for melanoma
WRRAPAPGA

SEQ ID NO: 26 - protein, 9 aa, Artificial Sequence
  tumour antigen for melanoma
PVTWRRAPA

SEQ ID NO: 27 - protein, 10 aa, Artificial Sequence
  tumour antigen for renal cell carcinoma, for melanoma andr enal cell carcinoma
SLFEGIDIYT

SEQ ID NO: 28 - protein, 9 aa, Artificial Sequence
  tumour antigen for bladder tumour
AEPINIQTW

SEQ ID NO: 29 - protein, 11 aa, Artificial Sequence
  tumour antigen for melanoma
FLEGNEVGKTY

SEQ ID NO: 30 - protein, 9 aa, Artificial Sequence
  tumour antigen for non-small cell lung carcinoma
FLDEFMEGV

SEQ ID NO: 31 - protein, 9 aa, Artificial Sequence
  tumour antigen for melanoma
EEKLIVVLF

SEQ ID NO: 32 - protein, 11 aa, Artificial Sequence
  tumour antigen for melanoma
SELFRSGLDSY

SEQ ID NO: 33 - protein, 9 aa, Artificial Sequence
  tumour antigen for melanoma
FRSGLDSYV

SEQ ID NO: 34 - protein, 9 aa, Artificial Sequence
  tumour antigen for melanoma
EAFIQPITR

SEQ ID NO: 35 - protein, 11 aa, Artificial Sequence
  tumour antigen for melanoma
RVIKNSIRLTL

SEQ ID NO: 36 - protein, 9 aa, Artificial Sequence
  tumour antigen for melanoma
KINKNPKYK

SEQ ID NO: 37 - protein, 8 aa, Artificial Sequence
  tumour antigen for lung squamous cell carcinoma
QQITKTEV

SEQ ID NO: 38 - protein, 10 aa, Artificial Sequence
  tumour antigen for colorectal carcinoma
SLYKFSPFPL

SEQ ID NO: 39 - protein, 9 aa, Artificial Sequence
  tumour antigen for melanoma
KELEGILLL

SEQ ID NO: 40 - protein, 9 aa, Artificial Sequence
  tumour antigen for head and neck squamous cell carcinoma
VVPCEPPEV

SEQ ID NO: 41 - protein, 25 aa, Artificial Sequence
  tumour antigen for promyelocytic leukemia
NSNHVASGAGEAAIETQSSSSEEIV

SEQ ID NO: 42 - protein, 10 aa, Artificial Sequence
  tumour antigen for melanoma
LLLDDLLVSI

SEQ ID NO: 43 - protein, 16 aa, Artificial Sequence
  tumour antigen for melanoma
PYYFAAELPPRNLPEP

SEQ ID NO: 44 - protein, 9 aa, Artificial Sequence
  tumour antigen for pancreatic adenocarcinoma
VVVGAVGVG

SEQ ID NO: 45 - protein, 10 aa, Artificial Sequence
  tumour antigen for melanoma
ILDTAGREEY

SEQ ID NO: 46 - protein, 9 aa, Artificial Sequence
  tumour antigen for melanoma
RPHVPESAF

SEQ ID NO: 47 - protein, 9 aa, Artificial Sequence
  tumour antigen for melanoma
KIFSEVTLK

SEQ ID NO: 48 - protein, 9 aa, Artificial Sequence
  tumour antigen for melanoma
SHETVIIEL

SEQ ID NO: 49 - protein, 10 aa, Artificial Sequence
  tumour antigen for sarcoma
QRPYGYDQIM

SEQ ID NO: 50 - protein, 9 aa, Artificial Sequence
  tumour antigen for colorectal carcinoma
RLSSCVPVA

SEQ ID NO: 51 - protein, 15 aa, Artificial Sequence
  tumour antigen for melanoma
GELIGILNAAKVPAD

SEQ ID NO: 52 - protein, 9 aa, Artificial Sequence
  shared tumour antigen
AARAVFLAL

SEQ ID NO: 53 - protein, 8 aa, Artificial Sequence
  shared tumour antigen
YRPRPRRY

SEQ ID NO: 54 - protein, 9 aa, Artificial Sequence
  shared tumour antigen
YYWPRPRRY

SEQ ID NO: 55 - protein, 10 aa, Artificial Sequence
  shared tumour antigen
VLPDVFIRCV

SEQ ID NO: 56 - protein, 9 aa, Artificial Sequence
  shared tumour antigen
MLAVISCAV

SEQ ID NO: 57 - protein, 9 aa, Artificial Sequence
  shared tumour antigen
RQKRILVNL

SEQ ID NO: 58 - protein, 9 aa, Artificial Sequence
  shared tumour antigen
NYNNFYRFL

SEQ ID NO: 59 - protein, 10 aa, Artificial Sequence
  shared tumour antigen
EYSKECLKEF

SEQ ID NO: 60 - protein, 9 aa, Artificial Sequence
  shared tumour antigen
EYLSLSDKI

SEQ ID NO: 61 - protein, 11 aa, Artificial Sequence
  shared tumour antigen
MLMAQEALAFL

SEQ ID NO: 62 - protein, 9 aa, Artificial Sequence
  shared tumour antigen
SLLMWITQC

SEQ ID NO: 63 - protein, 10 aa, Artificial Sequence
  shared tumour antigen
LAAQERRVPR

SEQ ID NO: 64 - protein, 9 aa, Artificial Sequence
  shared tumour antigen
ELVRRILSR

SEQ ID NO: 65 - protein, 9 aa, Artificial Sequence
  shared tumour antigen
APRGVRMAV

SEQ ID NO: 66 - protein, 14 aa, Artificial Sequence
  shared tumour antigen
SLLMWITQCFLPVF

SEQ ID NO: 67 - protein, 20 aa, Artificial Sequence
  shared tumour antigen
QGAMLAAQERRVPRAAEVPR

SEQ ID NO: 68 - protein, 18 aa, Artificial Sequence
  shared tumour antigen
AADHRQLQLSISSCLQQL

SEQ ID NO: 69 - protein, 22 aa, Artificial Sequence
  shared tumour antigen
CLSRRPWKRSWSAGSCPGMPHL

SEQ ID NO: 70 - protein, 22 aa, Artificial Sequence
  shared tumour antigen
CLSRRPWKRSWSAGSCPGMPHL

SEQ ID NO: 71 - protein, 13 aa, Artificial Sequence
  shared tumour antigen
ILSRDAAPLPRPG

SEQ ID NO: 72 - protein, 14 aa, Artificial Sequence
  shared tumour antigen
AGATGGRGPRGAGA

SEQ ID NO: 73 - protein, 9 aa, Artificial Sequence
  shared tumour antigen
EADPTGHSY

SEQ ID NO: 74 - protein, 9 aa, Artificial Sequence
  shared tumour antigen
KVLEYVIKV

SEQ ID NO: 75 - protein, 9 aa, Artificial Sequence
  shared tumour antigen
SLFRAVITK

SEQ ID NO: 76 - protein, 10 aa, Artificial Sequence
  shared tumour antigen
EVYDGREHSA

SEQ ID NO: 77 - protein, 9 aa, Artificial Sequence
  shared tumour antigen
RVRFFFPSL

SEQ ID NO: 78 - protein, 9 aa, Artificial Sequence
  shared tumour antigen
EADPTGHSY

SEQ ID NO: 79 - protein, 10 aa, Artificial Sequence
  shared tumour antigen
REPVTKAEML

SEQ ID NO: 80 - protein, 9 aa, Artificial Sequence
  shared tumour antigen
DPARYEFLW

SEQ ID NO: 81 - protein, 11 aa, Artificial Sequence
  shared tumour antigen
ITKKVADLVGF

SEQ ID NO: 82 - protein, 9 aa, Artificial Sequence
  shared tumour antigen
SAFPTTINF

SEQ ID NO: 83 - protein, 9 aa, Artificial Sequence
  shared tumour antigen
SAYGEPRKL

SEQ ID NO: 84 - protein, 9 aa, Artificial Sequence
  shared tumour antigen
SAYGEPRKL

SEQ ID NO: 85 - protein, 15 aa, Artificial Sequence
  shared tumour antigen
TSCILESLFRAVITK

SEQ ID NO: 86 - protein, 15 aa, Artificial Sequence
  shared tumour antigen
PRALAETSYVKVLEY

SEQ ID NO: 87 - protein, 16 aa, Artificial Sequence
  shared tumour antigen
FLLLKYRAREPVTKAE

SEQ ID NO: 88 - protein, 12 aa, Artificial Sequence
  shared tumour antigen
EYVIKVSARVRF

SEQ ID NO: 89 - protein, 10 aa, Artificial Sequence
  shared tumour antigen
YLQLVFGIEV

SEQ ID NO: 90 - protein, 9 aa, Artificial Sequence
  shared tumour antigen
EYLQLVFGI

SEQ ID NO: 91 - protein, 10 aa, Artificial Sequence
  shared tumour antigen
REPVTKAEML

SEQ ID NO: 92 - protein, 9 aa, Artificial Sequence
  shared tumour antigen
EGDCAPEEK

SEQ ID NO: 93 - protein, 14 aa, Artificial Sequence
  shared tumour antigen
LLKYRAREPVTKAE

SEQ ID NO: 94 - protein, 9 aa, Artificial Sequence
  shared tumour antigen
EVDPIGHLY

SEQ ID NO: 95 - protein, 9 aa, Artificial Sequence
  shared tumour antigen
FLWGPRALV

SEQ ID NO: 96 - protein, 9 aa, Artificial Sequence
  shared tumour antigen
KVAELVHFL

SEQ ID NO: 97 - protein, 9 aa, Artificial Sequence
  shared tumour antigen
TFPDLESEF

SEQ ID NO: 98 - protein, 9 aa, Artificial Sequence
  shared tumour antigen
VAELVHFLL

SEQ ID NO: 99 - protein, 10 aa, Artificial Sequence
  shared tumour antigen
MEVDPIGHLY

SEQ ID NO: 100 - protein, 9 aa, Artificial Sequence
  shared tumour antigen
EVDPIGHLY

SEQ ID NO: 101 - protein, 10 aa, Artificial Sequence
  shared tumour antigen
REPVTKAEML

SEQ ID NO: 102 - protein, 9 aa, Artificial Sequence
  shared tumour antigen
AELVHFLLL

SEQ ID NO: 103 - protein, 10 aa, Artificial Sequence
  shared tumour antigen
MEVDPIGHLY

SEQ ID NO: 104 - protein, 9 aa, Artificial Sequence
  shared tumour antigen
WQYFFPVIF

SEQ ID NO: 105 - protein, 9 aa, Artificial Sequence
  shared tumour antigen
EGDCAPEEK

SEQ ID NO: 106 - protein, 16 aa, Artificial Sequence
  shared tumour antigen
KKLLTQHFVQENYLEY

SEQ ID NO: 107 - protein, 16 aa, Artificial Sequence
  shared tumour antigen
KKLLTQHFVQENYLEY

SEQ ID NO: 108 - protein, 16 aa, Artificial Sequence
  shared tumour antigen
ACYEFLWGPRALVETS

SEQ ID NO: 109 - protein, 12 aa, Artificial Sequence
  shared tumour antigen
VIFSKASSSLQL

SEQ ID NO: 110 - protein, 12 aa, Artificial Sequence
  shared tumour antigen
VIFSKASSSLQL

SEQ ID NO: 111 - protein, 15 aa, Artificial Sequence
  shared tumour antigen
GDNQIMPKAGLLIIV

SEQ ID NO: 112 - protein, 15 aa, Artificial Sequence
  shared tumour antigen
TSYVKVLHHMVKISG

SEQ ID NO: 113 - protein, 16 aa, Artificial Sequence
  shared tumour antigen
RKVAELVHFLLLKYRA

SEQ ID NO: 114 - protein, 16 aa, Artificial Sequence
  shared tumour antigen
FLLLKYRAREPVTKAE

SEQ ID NO: 115 - protein, 9 aa, Artificial Sequence
  shared tumour antigen
EVDPASNTY

SEQ ID NO: 116 - protein, 10 aa, Artificial Sequence
  shared tumour antigen
GVYDGREHTV

SEQ ID NO: 117 - protein, 9 aa, Artificial Sequence
  shared tumour antigen
NYKRCFPVI

SEQ ID NO: 118 - protein, 8 aa, Artificial Sequence
  shared tumour antigen
SESLKMIF

SEQ ID NO: 119 - protein, 9 aa, Artificial Sequence
  shared tumour antigen
MVKISGGPR

SEQ ID NO: 120 - protein, 9 aa, Artificial Sequence
  shared tumour antigen
EVDPIGHVY

SEQ ID NO: 121 - protein, 10 aa, Artificial Sequence
  shared tumour antigen
REPVTKAEML

SEQ ID NO: 122 - protein, 9 aa, Artificial Sequence
  shared tumour antigen
EGDCAPEEK

SEQ ID NO: 123 - protein, 9 aa, Artificial Sequence
  shared tumour antigen
ISGGPRISY

SEQ ID NO: 124 - protein, 14 aa, Artificial Sequence
  shared tumour antigen
LLKYRAREPVTKAE

SEQ ID NO: 125 - protein, 9 aa, Artificial Sequence
  shared tumour antigen
ALSVMGVYV

SEQ ID NO: 126 - protein, 9 aa, Artificial Sequence
  shared tumour antigen
GLYDGMEHL

SEQ ID NO: 127 - protein, 9 aa, Artificial Sequence
  shared tumour antigen
DPARYEFLW

SEQ ID NO: 128 - protein, 9 aa, Artificial Sequence
  shared tumour antigen
FLWGPRALV

SEQ ID NO: 129 - protein, 9 aa, Artificial Sequence
  shared tumour antigen
VRIGHLYIL

SEQ ID NO: 130 - protein, 9 aa, Artificial Sequence
  shared tumour antigen
EGDCAPEEK

SEQ ID NO: 131 - protein, 15 aa, Artificial Sequence
  shared tumour antigen
REPFTKAEMLGSVIR

SEQ ID NO: 132 - protein, 14 aa, Artificial Sequence
  shared tumour antigen
AELVHFLLLKYRAR

SEQ ID NO: 133 - protein, 10 aa, Artificial Sequence
  shared tumour antigen
LLFGLALIEV

SEQ ID NO: 134 - protein, 9 aa, Artificial Sequence
  shared tumour antigen
ALKDVEERV

SEQ ID NO: 135 - protein, 9 aa, Artificial Sequence
  shared tumour antigen
SESIKKKVL

SEQ ID NO: 136 - protein, 20 aa, Artificial Sequence
  shared tumour antigen
PDTRPAPGSTAPPAHGVTSA

SEQ ID NO: 137 - protein, 9 aa, Artificial Sequence
  shared tumour antigen
QGQHFLQKV

SEQ ID NO: 138 - protein, 9 aa, Artificial Sequence
  shared tumour antigen
SLLMWITQC

SEQ ID NO: 139 - protein, 11 aa, Artificial Sequence
  shared tumour antigen
MLMAQEALAFL

SEQ ID NO: 140 - protein, 10 aa, Artificial Sequence
  shared tumour antigen
ASGPGGGAPR

SEQ ID NO: 141 - protein, 10 aa, Artificial Sequence
  shared tumour antigen
LAAQERRVPR

SEQ ID NO: 142 - protein, 10 aa, Artificial Sequence
  shared tumour antigen
TVSGNILTIR

SEQ ID NO: 143 - protein, 13 aa, Artificial Sequence
  shared tumour antigen
APRGPHGGAASGL

SEQ ID NO: 144 - protein, 9 aa, Artificial Sequence
  shared tumour antigen
MPFATPMEA

SEQ ID NO: 145 - protein, 12 aa, Artificial Sequence
  shared tumour antigen
KEFTVSGNILTI

SEQ ID NO: 146 - protein, 9 aa, Artificial Sequence
  shared tumour antigen
MPFATPMEA

SEQ ID NO: 147 - protein, 9 aa, Artificial Sequence
  shared tumour antigen
LAMPFATPM

SEQ ID NO: 148 - protein, 9 aa, Artificial Sequence
  shared tumour antigen
ARGPESRLL

SEQ ID NO: 149 - protein, 14 aa, Artificial Sequence
  shared tumour antigen
SLLMWITQCFLPVF

SEQ ID NO: 150 - protein, 25 aa, Artificial Sequence
  shared tumour antigen
LLEFYLAMPFATPMEAELARRSLAQ

SEQ ID NO: 151 - protein, 25 aa, Artificial Sequence
  shared tumour antigen
LLEFYLAMPFATPMEAELARRSLAQ

SEQ ID NO: 152 - protein, 12 aa, Artificial Sequence
  shared tumour antigen
EFYLAMPFATPM

SEQ ID NO: 153 - protein, 12 aa, Artificial Sequence
  shared tumour antigen
RLLEFYLAMPFA

SEQ ID NO: 154 - protein, 20 aa, Artificial Sequence
  shared tumour antigen
QGAMLAAQERRVPRAAEVPR

SEQ ID NO: 155 - protein, 20 aa, Artificial Sequence
  shared tumour antigen
PGVLLKEFTVSGNILTIRLT

SEQ ID NO: 156 - protein, 10 aa, Artificial Sequence
  shared tumour antigen
VLLKEFTVSG

SEQ ID NO: 157 - protein, 18 aa, Artificial Sequence
  shared tumour antigen
AADHRQLQLSISSCLQQL

SEQ ID NO: 158 - protein, 25 aa, Artificial Sequence
  shared tumour antigen
LLEFYLAMPFATPMEAELARRSLAQ

SEQ ID NO: 159 - protein, 15 aa, Artificial Sequence
  shared tumour antigen
LKEFTVSGNILTIRL

SEQ ID NO: 160 - protein, 25 aa, Artificial Sequence
  shared tumour antigen
PGVLLKEFTVSGNILTIRLTAADHR

SEQ ID NO: 161 - protein, 25 aa, Artificial Sequence
  shared tumour antigen
LLEFYLAMPFATPMEAELARRSLAQ

SEQ ID NO: 162 - protein, 14 aa, Artificial Sequence
  shared tumour antigen
AGATGGRGPRGAGA

SEQ ID NO: 163 - protein, 9 aa, Artificial Sequence
  shared tumour antigen
LYATVIHDI

SEQ ID NO: 164 - protein, 9 aa, Artificial Sequence
  shared tumour antigen
ILDSSEEDK

SEQ ID NO: 165 - protein, 9 aa, Artificial Sequence
  shared tumour antigen
KASEKIFYV

SEQ ID NO: 166 - protein, 16 aa, Artificial Sequence
  shared tumour antigen
EKIQKAFDDIAKYFSK

SEQ ID NO: 167 - protein, 18 aa, Artificial Sequence
  shared tumour antigen
WEKMKASEKIFYVYMKRK

SEQ ID NO: 168 - protein, 15 aa, Artificial Sequence
  shared tumour antigen
KIFYVYMKRKYEAMT

SEQ ID NO: 169 - protein, 14 aa, Artificial Sequence
  shared tumour antigen
KIFYVYMKRKYEAM

SEQ ID NO: 170 - protein, 20 aa, Artificial Sequence
  shared tumour antigen
INKTSGPKRGKHAWTHRLRE

SEQ ID NO: 171 - protein, 20 aa, Artificial Sequence
  shared tumour antigen
YFSKKEWEKMKSSEKIVYVY

SEQ ID NO: 172 - protein, 20 aa, Artificial Sequence
  shared tumour antigen
MKLNYEVMTKLGFKVTLPPF

SEQ ID NO: 173 - protein, 20 aa, Artificial Sequence
  shared tumour antigen
KHAWTHRLRERKQLVVYEEI

SEQ ID NO: 174 - protein, 20 aa, Artificial Sequence
  shared tumour antigen
LGFKVTLPPFMRSKRAADFH

SEQ ID NO: 175 - protein, 20 aa, Artificial Sequence
  shared tumour antigen
KSSEKIVYVYMKLNYEVMTK

SEQ ID NO: 176 - protein, 20 aa, Artificial Sequence
  shared tumour antigen
KHAWTHRLRERKQLVVYEEI

SEQ ID NO: 177 - protein, 9 aa, Artificial Sequence
  shared tumour antigen
SLGWLFLLL

SEQ ID NO: 178 - protein, 9 aa, Artificial Sequence
  shared tumour antigen
LSRLSNRLL

SEQ ID NO: 179 - protein, 9 aa, Artificial Sequence
  shared tumour antigen
LSRLSNRLL

SEQ ID NO: 180 - protein, 15 aa, Artificial Sequence
  shared tumour antigen
CEFHACWPAFTVLGE

SEQ ID NO: 181 - protein, 15 aa, Artificial Sequence
  shared tumour antigen
CEFHACWPAFTVLGE

SEQ ID NO: 182 - protein, 15 aa, Artificial Sequence
  shared tumour antigen
CEFHACWPAFTVLGE

SEQ ID NO: 183 - protein, 10 aa, Artificial Sequence
  shared tumour antigen
EVISCKLIKR

SEQ ID NO: 184 - protein, 17 aa, Artificial Sequence
  shared tumour antigen
CATWKVICKSCISQTPG

SEQ ID NO: 185 - protein, 9 aa, Artificial Sequence
  tumour differentiation antigen
YLSGANLNL

SEQ ID NO: 186 - protein, 9 aa, Artificial Sequence
  tumour differentiation antigen
IMIGVLVGV

SEQ ID NO: 187 - protein, 9 aa, Artificial Sequence
  tumour differentiation antigen
GVLVGVALI

SEQ ID NO: 188 - protein, 9 aa, Artificial Sequence
  tumour differentiation antigen
HLFGYSWYK

SEQ ID NO: 189 - protein, 10 aa, Artificial Sequence
  tumour differentiation antigen
QYSWFVNGTF

SEQ ID NO: 190 - protein, 9 aa, Artificial Sequence
  tumour differentiation antigen
TYACFVSNL

SEQ ID NO: 191 - protein, 15 aa, Artificial Sequence
  tumour differentiation antigen
AYVCGIQNSVSANRS

SEQ ID NO: 192 - protein, 25 aa, Artificial Sequence
  tumour differentiation antigen
DTGFYTLHVIKSDLVNEEATGQFRV

SEQ ID NO: 193 - protein, 15 aa, Artificial Sequence
  tumour differentiation antigen
YSWRINGIPQQHTQV

SEQ ID NO: 194 - protein, 13 aa, Artificial Sequence
  tumour differentiation antigen
TYYRPGVNLSLSC

SEQ ID NO: 195 - protein, 13 aa, Artificial Sequence
  tumour differentiation antigen
EIIYPNASLLIQN

SEQ ID NO: 196 - protein, 15 aa, Artificial Sequence
  tumour differentiation antigen
YACFVSNLATGRNNS

SEQ ID NO: 197 - protein, 13 aa, Artificial Sequence
  tumour differentiation antigen
LWWVNNQSLPVSP

SEQ ID NO: 198 - protein, 13 aa, Artificial Sequence
  tumour differentiation antigen
LWWVNNQSLPVSP

SEQ ID NO: 199 - protein, 13 aa, Artificial Sequence
  tumour differentiation antigen
LWWVNNQSLPVSP

SEQ ID NO: 200 - protein, 13 aa, Artificial Sequence
  tumour differentiation antigen
EIIYPNASLLIQN

SEQ ID NO: 201 - protein, 13 aa, Artificial Sequence
  tumour differentiation antigen
NSIVKSITVSASG

SEQ ID NO: 202 - protein, 9 aa, Artificial Sequence
  tumour differentiation antigen
KTWGQYWQV

SEQ ID NO: 203 - protein, 10 aa, Artificial Sequence
  tumour differentiation antigen
AMLGTHTMEV

SEQ ID NO: 204 - protein, 9 aa, Artificial Sequence
  tumour differentiation antigen
ITDQVPFSV

SEQ ID NO: 205 - protein, 9 aa, Artificial Sequence
  tumour differentiation antigen
YLEPGPVTA

SEQ ID NO: 206 - protein, 10 aa, Artificial Sequence
  tumour differentiation antigen
LLDGTATLRL

SEQ ID NO: 207 - protein, 10 aa, Artificial Sequence
  tumour differentiation antigen
VLYRYGSFSV

SEQ ID NO: 208 - protein, 10 aa, Artificial Sequence
  tumour differentiation antigen
SLADTNSLAV

SEQ ID NO: 209 - protein, 9 aa, Artificial Sequence
  tumour differentiation antigen
RLMKQDFSV

SEQ ID NO: 210 - protein, 9 aa, Artificial Sequence
  tumour differentiation antigen
RLPRIFCSC

SEQ ID NO: 211 - protein, 9 aa, Artificial Sequence
  tumour differentiation antigen
LIYRRRLMK

SEQ ID NO: 212 - protein, 9 aa, Artificial Sequence
  tumour differentiation antigen
ALLAVGATK

SEQ ID NO: 213 - protein, 10 aa, Artificial Sequence
  tumour differentiation antigen
IALNFPGSQK

SEQ ID NO: 214 - protein, 9 aa, Artificial Sequence
  tumour differentiation antigen
ALNFPGSQK

SEQ ID NO: 215 - protein, 9 aa, Artificial Sequence
  tumour differentiation antigen
ALNFPGSQK

SEQ ID NO: 216 - protein, 9 aa, Artificial Sequence
  tumour differentiation antigen
VYFFLPDHL

SEQ ID NO: 217 - protein, 9 aa, Artificial Sequence
  tumour differentiation antigen
RTKQLYPEW

SEQ ID NO: 218 - protein, 10 aa, Artificial Sequence
  tumour differentiation antigen
HTMEVTVYHR

SEQ ID NO: 219 - protein, 9 aa, Artificial Sequence
  tumour differentiation antigen
SSPGCQPPA

SEQ ID NO: 220 - protein, 10 aa, Artificial Sequence
  tumour differentiation antigen
VPLDCVLYRY

SEQ ID NO: 221 - protein, 9 aa, Artificial Sequence
  tumour differentiation antigen
LPHSSSHWL

SEQ ID NO: 222 - protein, 8 aa, Artificial Sequence
  tumour differentiation antigen
SNDGPTLI

SEQ ID NO: 223 - protein, 15 aa, Artificial Sequence
  tumour differentiation antigen
GRAMLGTHTMEVTVY

SEQ ID NO: 224 - protein, 16 aa, Artificial Sequence
  tumour differentiation antigen
WNRQLYPEWTEAQRLD

SEQ ID NO: 225 - protein, 18 aa, Artificial Sequence
  tumour differentiation antigen
TTEWVETTARELPIPEPE

SEQ ID NO: 226 - protein, 17 aa, Artificial Sequence
  tumour differentiation antigen
TGRAMLGTHTMEVTVYH

SEQ ID NO: 227 - protein, 15 aa, Artificial Sequence
  tumour differentiation antigen
GRAMLGTHTMEVTVY

SEQ ID NO: 228 - protein, 15 aa, Artificial Sequence
  tumour differentiation antigen
SVSESDTIRSISIAS

SEQ ID NO: 229 - protein, 15 aa, Artificial Sequence
  tumour differentiation antigen
LLANGRMPTVLQCVN

SEQ ID NO: 230 - protein, 15 aa, Artificial Sequence
  tumour differentiation antigen
RMPTVLQCVNVSVVS

SEQ ID NO: 231 - protein, 9 aa, Artificial Sequence
  tumour differentiation antigen
PLLENVISK

SEQ ID NO: 232 - protein, 10 aa, Artificial Sequence
  tumour differentiation antigen
EAAGIGILTV

SEQ ID NO: 233 - protein, 9 aa, Artificial Sequence
  tumour differentiation antigen
ILTVILGVL

SEQ ID NO: 234 - protein, 10 aa, Artificial Sequence
  tumour differentiation antigen
EAAGIGILTV

SEQ ID NO: 235 - protein, 11 aa, Artificial Sequence
  tumour differentiation antigen
AEEAAGIGILT

SEQ ID NO: 236 - protein, 11 aa, Artificial Sequence
  tumour differentiation antigen
RNGYRALMDKS

SEQ ID NO: 237 - protein, 12 aa, Artificial Sequence
  tumour differentiation antigen
EEAAGIGILTVI

SEQ ID NO: 238 - protein, 14 aa, Artificial Sequence
  tumour differentiation antigen
AAGIGILTVILGVL

SEQ ID NO: 239 - protein, 13 aa, Artificial Sequence
  tumour differentiation antigen
APPAYEKLPSAEQ

SEQ ID NO: 240 - protein, 12 aa, Artificial Sequence
  tumour differentiation antigen
EEAAGIGILTVI

SEQ ID NO: 241 - protein, 23 aa, Artificial Sequence
  tumour differentiation antigen
RNGYRALMDKSLHVGTQCALTRR

SEQ ID NO: 242 - protein, 20 aa, Artificial Sequence
  tumour differentiation antigen
MPREDAHFIYGYPKKGHGHS

SEQ ID NO: 243 - protein, 20 aa, Artificial Sequence
  tumour differentiation antigen
KNCEPVVPNAPPAYEKLSAE

SEQ ID NO: 244 - protein, 9 aa, Artificial Sequence
  tumour differentiation antigen
SLSKILDTV

SEQ ID NO: 245 - protein, 9 aa, Artificial Sequence
  tumour differentiation antigen
LYSACFWWL

SEQ ID NO: 246 - protein, 10 aa, Artificial Sequence
  tumour differentiation antigen
FLTPKKLQCV

SEQ ID NO: 247 - protein, 10 aa, Artificial Sequence
  tumour differentiation antigen
VISNDVCAQV

SEQ ID NO: 248 - protein, 9 aa, Artificial Sequence
  tumour differentiation antigen
VLHWDPETV

SEQ ID NO: 249 - protein, 9 aa, Artificial Sequence
  tumour differentiation antigen
MSLQRQFLR

SEQ ID NO: 250 - protein, 21 aa, Artificial Sequence
  tumour differentiation antigen
ISPNSVFSQWRVVCDSLEDYD

SEQ ID NO: 251 - protein, 10 aa, Artificial Sequence
  tumour differentiation antigen
SLPYWNFATG

SEQ ID NO: 252 - protein, 9 aa, Artificial Sequence
  tumour differentiation antigen
SVYDFFVWL

SEQ ID NO: 253 - protein, 9 aa, Artificial Sequence
  tumour differentiation antigen
TLDSQVMSL

SEQ ID NO: 254 - protein, 9 aa, Artificial Sequence
  tumour differentiation antigen
LLGPGRPYR

SEQ ID NO: 255 - protein, 9 aa, Artificial Sequence
  tumour differentiation antigen
LLGPGRPYR

SEQ ID NO: 256 - protein, 9 aa, Artificial Sequence
  tumour differentiation antigen
ANDPIFVVL

SEQ ID NO: 257 - protein, 15 aa, Artificial Sequence
  tumour differentiation antigen
QCTEVRADTRPWSGP

SEQ ID NO: 258 - protein, 10 aa, Artificial Sequence
  tumour differentiation antigen
ALPYWNFATG

SEQ ID NO: 259 - protein, 9 aa, Artificial Sequence
  tumour differentiation antigen
KCDICTDEY

SEQ ID NO: 260 - protein, 11 aa, Artificial Sequence
  tumour differentiation antigen
SSDYVIPIGTY

SEQ ID NO: 261 - protein, 9 aa, Artificial Sequence
  tumour differentiation antigen
MLLAVLYCL

SEQ ID NO: 262 - protein, 10 aa, Artificial Sequence
  tumour differentiation antigen
CLLWSFQTSA

SEQ ID NO: 263 - protein, 9 aa, Artificial Sequence
  tumour differentiation antigen
YMDGTMSQV

SEQ ID NO: 264 - protein, 9 aa, Artificial Sequence
  tumour differentiation antigen
AFLPWHRLF

SEQ ID NO: 265 - protein, 9 aa, Artificial Sequence
  tumour differentiation antigen
QCSGNFMGF

SEQ ID NO: 266 - protein, 12 aa, Artificial Sequence
  tumour differentiation antigen
TPRLPSSADVEF

SEQ ID NO: 267 - protein, 9 aa, Artificial Sequence
  tumour differentiation antigen
LPSSADVEF

SEQ ID NO: 268 - protein, 10 aa, Artificial Sequence
  tumour differentiation antigen
LHHAFVDSIF

SEQ ID NO: 269 - protein, 9 aa, Artificial Sequence
  tumour differentiation antigen
SEIWRDIDF

SEQ ID NO: 270 - protein, 15 aa, Artificial Sequence
  tumour differentiation antigen
QNILLSNAPLGPQFP

SEQ ID NO: 271 - protein, 13 aa, Artificial Sequence
  tumour differentiation antigen
SYLQDSDPDSFQD

SEQ ID NO: 272 - protein, 21 aa, Artificial Sequence [sequence omitted]

SEQ ID NO: 273 - protein, 9 aa, Artificial Sequence
  antigen overexpressed in tumour
SVASTITGV

SEQ ID NO: 274 - protein, 10 aa, Artificial Sequence
  antigen overexpressed in tumour
RSDSGQQARY

SEQ ID NO: 275 - protein, 9 aa, Artificial Sequence
  antigen overexpressed in tumour
LLYKLADLI

SEQ ID NO: 276 - protein, 10 aa, Artificial Sequence
  antigen overexpressed in tumour
YLNDHLEPWI

SEQ ID NO: 277 - protein, 9 aa, Artificial Sequence
  antigen overexpressed in tumour
CQWGRLWQL

SEQ ID NO: 278 - protein, 10 aa, Artificial Sequence
  antigen overexpressed in tumour
VLLQAGSLHA

SEQ ID NO: 279 - protein, 9 aa, Artificial Sequence
  antigen overexpressed in tumour
KVHPVIWSL

SEQ ID NO: 280 - protein, 10 aa, Artificial Sequence
  antigen overexpressed in tumour
LMLQNALTTM

SEQ ID NO: 281 - protein, 9 aa, Artificial Sequence
  antigen overexpressed in tumour
LLGATCMFV

SEQ ID NO: 282 - protein, 15 aa, Artificial Sequence
  antigen overexpressed in tumour
NPPSMVAAGSVVAAV

SEQ ID NO: 283 - protein, 10 aa, Artificial Sequence
  antigen overexpressed in tumour
ALGGHPLLGV

SEQ ID NO: 284 - protein, 9 aa, Artificial Sequence
  antigen overexpressed in tumour
TMNGSKSPV

SEQ ID NO: 285 - protein, 9 aa, Artificial Sequence
  antigen overexpressed in tumour
RYQLDPKFI

SEQ ID NO: 286 - protein, 12 aa, Artificial Sequence
  antigen overexpressed in tumour
DVTFNIICKKCG

SEQ ID NO: 287 - protein, 9 aa, Artificial Sequence
  antigen overexpressed in tumour
FMVEDETVL

SEQ ID NO: 288 - protein, 10 aa, Artificial Sequence
  antigen overexpressed in tumour
FINDEIFVEL

SEQ ID NO: 289 - protein, 9 aa, Artificial Sequence
  antigen overexpressed in tumour
KYDCFLHPF

SEQ ID NO: 290 - protein, 9 aa, Artificial Sequence
  antigen overexpressed in tumour
KYVGIEREM

SEQ ID NO: 291 - protein, 9 aa, Artificial Sequence
  antigen overexpressed in tumour
NTYASPRFK

SEQ ID NO: 292 - protein, 9 aa, Artificial Sequence
  antigen overexpressed in tumour
HLSTAFARV

SEQ ID NO: 293 - protein, 9 aa, Artificial Sequence
  antigen overexpressed in tumour
KIFGSLAFL

SEQ ID NO: 294 - protein, 9 aa, Artificial Sequence
  antigen overexpressed in tumour
IISAVVGIL

SEQ ID NO: 295 - protein, 9 aa, Artificial Sequence
  antigen overexpressed in tumour
ALCRWGLLL

SEQ ID NO: 296 - protein, 9 aa, Artificial Sequence
  antigen overexpressed in tumour
ILHNGAYSL

SEQ ID NO: 297 - protein, 9 aa, Artificial Sequence
  antigen overexpressed in tumour
RLLQETELV

SEQ ID NO: 298 - protein, 9 aa, Artificial Sequence
  antigen overexpressed in tumour
VVLGVVFGI

SEQ ID NO: 299 - protein, 10 aa, Artificial Sequence
  antigen overexpressed in tumour
YMIMVKCWMI

SEQ ID NO: 300 - protein, 9 aa, Artificial Sequence
  antigen overexpressed in tumour
HLYQGCQVV

SEQ ID NO: 301 - protein, 10 aa, Artificial Sequence
  antigen overexpressed in tumour
YLVPQQGFFC

SEQ ID NO: 302 - protein, 9 aa, Artificial Sequence
  antigen overexpressed in tumour
PLQPEQLQV

SEQ ID NO: 303 - protein, 9 aa, Artificial Sequence
  antigen overexpressed in tumour
TLEEITGYL

SEQ ID NO: 304 - protein, 9 aa, Artificial Sequence
  antigen overexpressed in tumour
ALIHHNTHL

SEQ ID NO: 305 - protein, 9 aa, Artificial Sequence
  antigen overexpressed in tumour
PLTSIISAV

SEQ ID NO: 306 - protein, 9 aa, Artificial Sequence
  antigen overexpressed in tumour
VLRENTSPK

SEQ ID NO: 307 - protein, 9 aa, Artificial Sequence
  antigen overexpressed in tumour
TYLPTNASL

SEQ ID NO: 308 - protein, 9 aa, Artificial Sequence
  antigen overexpressed in tumour
ALLEIASCL

SEQ ID NO: 309 - protein, 9 aa, Artificial Sequence
  antigen overexpressed in tumour
WLPFGFILI

SEQ ID NO: 310 - protein, 9 aa, Artificial Sequence
  antigen overexpressed in tumour
SPRWWPTCL

SEQ ID NO: 311 - protein, 9 aa, Artificial Sequence
  antigen overexpressed in tumour
GVALQTMKQ

SEQ ID NO: 312 - protein, 9 aa, Artificial Sequence
  antigen overexpressed in tumour
FMNKFIYEI

SEQ ID NO: 313 - protein, 10 aa, Artificial Sequence
  antigen overexpressed in tumour
QLAVSVILRV

SEQ ID NO: 314 - protein, 14 aa, Artificial Sequence
  antigen overexpressed in tumour
LPAVVGLSPGEQEY

SEQ ID NO: 315 - protein, 16 aa, Artificial Sequence
  antigen overexpressed in tumour
VGQDVSVLFRVTGALQ

SEQ ID NO: 316 - protein, 8 aa, Artificial Sequence
  antigen overexpressed in tumour
VLFYLGQY

SEQ ID NO: 317 - protein, 9 aa, Artificial Sequence
  antigen overexpressed in tumour
TLNDECWPA

SEQ ID NO: 318 - protein, 9 aa, Artificial Sequence
  antigen overexpressed in tumour
GLPPDVQRV

SEQ ID NO: 319 - protein, 12 aa, Artificial Sequence
  antigen overexpressed in tumour
SLFPNSPKWTSK

SEQ ID NO: 320 - protein, 9 aa, Artificial Sequence
  antigen overexpressed in tumour
STAPPVHNV

SEQ ID NO: 321 - protein, 9 aa, Artificial Sequence
  antigen overexpressed in tumour
LLLLTVLTV

SEQ ID NO: 322 - protein, 12 aa, Artificial Sequence
  antigen overexpressed in tumour
PGSTAPPAHGVT

SEQ ID NO: 323 - protein, 9 aa, Artificial Sequence
  antigen overexpressed in tumour
LLGRNSFEV

SEQ ID NO: 324 - protein, 9 aa, Artificial Sequence
  antigen overexpressed in tumour
RMPEAAPPV

SEQ ID NO: 325 - protein, 9 aa, Artificial Sequence
  antigen overexpressed in tumour
SQKTYQGSY

SEQ ID NO: 326 - protein, 13 aa, Artificial Sequence
  antigen overexpressed in tumour
PGTRVRAMAIYKQ

SEQ ID NO: 327 - protein, 12 aa, Artificial Sequence
  antigen overexpressed in tumour
HLIRVEGNLRVE

SEQ ID NO: 328 - protein, 9 aa, Artificial Sequence
  antigen overexpressed in tumour
TLPGYPPHV

SEQ ID NO: 329 - protein, 12 aa, Artificial Sequence
  antigen overexpressed in tumour
CTACRWKKACQR

SEQ ID NO: 330 - protein, 9 aa, Artificial Sequence
  antigen overexpressed in tumour
VLDGLDVLL

SEQ ID NO: 331 - protein, 10 aa, Artificial Sequence
  antigen overexpressed in tumour
SLYSFPEPEA

SEQ ID NO: 332 - protein, 10 aa, Artificial Sequence
  antigen overexpressed in tumour
ALYVDSLFFL

SEQ ID NO: 333 - protein, 9 aa, Artificial Sequence
  antigen overexpressed in tumour
SLLQHLIGL

SEQ ID NO: 334 - protein, 9 aa, Artificial Sequence
  antigen overexpressed in tumour
LYVDSLFFL

SEQ ID NO: 335 - protein, 9 aa, Artificial Sequence
  antigen overexpressed in tumour
NYARTEDFF

SEQ ID NO: 336 - protein, 9 aa, Artificial Sequence
  antigen overexpressed in tumour
LKLSGVVRL

SEQ ID NO: 337 - protein, 10 aa, Artificial Sequence
  antigen overexpressed in tumour
PLPPARNGGL

SEQ ID NO: 338 - protein, 10 aa, Artificial Sequence
  antigen overexpressed in tumour
SPSSNRIRNT

SEQ ID NO: 339 - protein, 9 aa, Artificial Sequence
  antigen overexpressed in tumour
LAALPHSCL

SEQ ID NO: 340 - protein, 10 aa, Artificial Sequence
  antigen overexpressed in tumour
GLASFKSFLK

SEQ ID NO: 341 - protein, 10 aa, Artificial Sequence
  antigen overexpressed in tumour
RAGLQVRKNK

SEQ ID NO: 342 - protein, 10 aa, Artificial Sequence
  antigen overexpressed in tumour
ALWPWLLMAT

SEQ ID NO: 343 - protein, 9 aa, Artificial Sequence
  antigen overexpressed in tumour
NSQPVWLCL

SEQ ID NO: 344 - protein, 9 aa, Artificial Sequence
  antigen overexpressed in tumour
LPRWPPPQL

SEQ ID NO: 345 - protein, 9 aa, Artificial Sequence
  antigen overexpressed in tumour
KMDAEHPEL

SEQ ID NO: 346 - protein, 9 aa, Artificial Sequence
  antigen overexpressed in tumour
AWISKPPGV

SEQ ID NO: 347 - protein, 10 aa, Artificial Sequence
  antigen overexpressed in tumour
SAWISKPPGV

SEQ ID NO: 348 - protein, 9 aa, Artificial Sequence
  antigen overexpressed in tumour
MIAVFLPIV

SEQ ID NO: 349 - protein, 15 aa, Artificial Sequence
  antigen overexpressed in tumour
HQQYFYKIPILVINK

SEQ ID NO: 350 - protein, 10 aa, Artificial Sequence
  antigen overexpressed in tumour
ELTLGEFLKL

SEQ ID NO: 351 - protein, 9 aa, Artificial Sequence
  antigen overexpressed in tumour
ILAKFLHWL

SEQ ID NO: 352 - protein, 9 aa, Artificial Sequence
  antigen overexpressed in tumour
RLVDDFLLV

SEQ ID NO: 353 - protein, 15 aa, Artificial Sequence
  antigen overexpressed in tumour
RPGLLGASVLGLDDI

SEQ ID NO: 354 - protein, 15 aa, Artificial Sequence
  antigen overexpressed in tumour
LTDLQPYMRQFVAHL

SEQ ID NO: 355 - protein, 9 aa, Artificial Sequence
  antigen overexpressed in tumour
SRFGGAVVR

SEQ ID NO: 356 - protein, 11 aa, Artificial Sequence
  antigen overexpressed in tumour
TSEKRPFMCAY

SEQ ID NO: 357 - protein, 9 aa, Artificial Sequence
  antigen overexpressed in tumour
CMTWNQMNL

SEQ ID NO: 358 - protein, 11 aa, Artificial Sequence
  antigen overexpressed in tumour
LSHLQMHSRKH

SEQ ID NO: 359 - protein, 16 aa, Artificial Sequence
  antigen overexpressed in tumour
KRYFKLSHLQMHSRKH

SEQ ID NO: 360 - protein, 10 aa, Artificial Sequence
  CHAIN 1..10: Ndufs1
  tumour antigen
AAVSNMVQKI